SEQUENCE LISTING (140 sequences)

SEQ ID NO: 1 - protein, 16 aa, Artificial Sequence
  Description of Artificial Sequence Synthetic peptide
TFVYGGCRAKRNNFKS

SEQ ID NO: 2 - protein, 19 aa, Artificial Sequence
  Description of Artificial Sequence Synthetic peptide
TFQYGGCMGNGNNFVTEKE

SEQ ID NO: 3 - protein, 19 aa, Artificial Sequence
  Description of Artificial Sequence Synthetic peptide
PFFYGGCGGNRNNFDTEEY

SEQ ID NO: 4 - protein, 19 aa, Artificial Sequence
  Description of Artificial Sequence Synthetic peptide
SFYYGGCLGNKNNYLREEE

SEQ ID NO: 5 - protein, 19 aa, Artificial Sequence
  Description of Artificial Sequence Synthetic peptide
TFFYGGCRAKRNNFKRAKY

SEQ ID NO: 6 - protein, 19 aa, Artificial Sequence
  Description of Artificial Sequence Synthetic peptide
TFFYGGCRGKRNNFKRAKY

SEQ ID NO: 7 - protein, 19 aa, Artificial Sequence
  Description of Artificial Sequence Synthetic peptide
TFFYGGCRAKKNNYKRAKY

SEQ ID NO: 8 - protein, 19 aa, Artificial Sequence
  Description of Artificial Sequence Synthetic peptide
TFFYGGCRGKKNNFKRAKY

SEQ ID NO: 9 - protein, 19 aa, Artificial Sequence
  Description of Artificial Sequence Synthetic peptide
TFQYGGCRAKRNNFKRAKY

SEQ ID NO: 10 - protein, 19 aa, Artificial Sequence
  Description of Artificial Sequence Synthetic peptide
TFQYGGCRGKKNNFKRAKY

SEQ ID NO: 11 - protein, 19 aa, Artificial Sequence
  Description of Artificial Sequence Synthetic peptide
TFFYGGCLGKRNNFKRAKY

SEQ ID NO: 12 - protein, 19 aa, Artificial Sequence
  Description of Artificial Sequence Synthetic peptide
TFFYGGSLGKRNNFKRAKY

SEQ ID NO: 13 - protein, 19 aa, Artificial Sequence
  Description of Artificial Sequence Synthetic peptide
PFFYGGCGGKKNNFKRAKY

SEQ ID NO: 14 - protein, 19 aa, Artificial Sequence
  Description of Artificial Sequence Synthetic peptide
TFFYGGCRGKGNNYKRAKY

SEQ ID NO: 15 - protein, 19 aa, Artificial Sequence
  Description of Artificial Sequence Synthetic peptide
PFFYGGCRGKRNNFLRAKY

SEQ ID NO: 16 - protein, 19 aa, Artificial Sequence
  Description of Artificial Sequence Synthetic peptide
TFFYGGCRGKRNNFKREKY

SEQ ID NO: 17 - protein, 19 aa, Artificial Sequence
  Description of Artificial Sequence Synthetic peptide
PFFYGGCRAKKNNFKRAKE

SEQ ID NO: 18 - protein, 19 aa, Artificial Sequence
  Description of Artificial Sequence Synthetic peptide
TFFYGGCRGKRNNFKRAKD

SEQ ID NO: 19 - protein, 19 aa, Artificial Sequence
  Description of Artificial Sequence Synthetic peptide
TFFYGGCRAKRNNFDRAKY

SEQ ID NO: 20 - protein, 19 aa, Artificial Sequence
  Description of Artificial Sequence Synthetic peptide
TFFYGGCRGKKNNFKRAEY

SEQ ID NO: 21 - protein, 19 aa, Artificial Sequence
  Description of Artificial Sequence Synthetic peptide
PFFYGGCGANRNNFKRAKY

SEQ ID NO: 22 - protein, 19 aa, Artificial Sequence
  Description of Artificial Sequence Synthetic peptide
TFFYGGCGGKKNNFKTAKY

SEQ ID NO: 23 - protein, 19 aa, Artificial Sequence
  Description of Artificial Sequence Synthetic peptide
TFFYGGCRGNRNNFLRAKY

SEQ ID NO: 24 - protein, 19 aa, Artificial Sequence
  Description of Artificial Sequence Synthetic peptide
TFFYGGCRGNRNNFKTAKY

SEQ ID NO: 25 - protein, 19 aa, Artificial Sequence
  Description of Artificial Sequence Synthetic peptide
TFFYGGSRGNRNNFKTAKY

SEQ ID NO: 26 - protein, 19 aa, Artificial Sequence
  Description of Artificial Sequence Synthetic peptide
TFFYGGCLGNGNNFKRAKY

SEQ ID NO: 27 - protein, 19 aa, Artificial Sequence
  Description of Artificial Sequence Synthetic peptide
TFFYGGCLGNRNNFLRAKY

SEQ ID NO: 28 - protein, 19 aa, Artificial Sequence
  Description of Artificial Sequence Synthetic peptide
TFFYGGCLGNRNNFKTAKY

SEQ ID NO: 29 - protein, 19 aa, Artificial Sequence
  Description of Artificial Sequence Synthetic peptide
TFFYGGCRGNGNNFKSAKY

SEQ ID NO: 30 - protein, 19 aa, Artificial Sequence
  Description of Artificial Sequence Synthetic peptide
TFFYGGCRGKKNNFDREKY

SEQ ID NO: 31 - protein, 19 aa, Artificial Sequence
  Description of Artificial Sequence Synthetic peptide
TFFYGGCRGKRNNFLREKE

SEQ ID NO: 32 - protein, 19 aa, Artificial Sequence
  Description of Artificial Sequence Synthetic peptide
TFFYGGCRGKGNNFDRAKY

SEQ ID NO: 33 - protein, 19 aa, Artificial Sequence
  Description of Artificial Sequence Synthetic peptide
TFFYGGSRGKGNNFDRAKY

SEQ ID NO: 34 - protein, 19 aa, Artificial Sequence
  Description of Artificial Sequence Synthetic peptide
TFFYGGCRGNGNNFVTAKY

SEQ ID NO: 35 - protein, 19 aa, Artificial Sequence
  Description of Artificial Sequence Synthetic peptide
PFFYGGCGGKGNNYVTAKY

SEQ ID NO: 36 - protein, 19 aa, Artificial Sequence
  Description of Artificial Sequence Synthetic peptide
TFFYGGCLGKGNNFLTAKY

SEQ ID NO: 37 - protein, 19 aa, Artificial Sequence
  Description of Artificial Sequence Synthetic peptide
SFFYGGCLGNKNNFLTAKY

SEQ ID NO: 38 - protein, 19 aa, Artificial Sequence
  Description of Artificial Sequence Synthetic peptide
TFFYGGCGGNKNNFVREKY

SEQ ID NO: 39 - protein, 19 aa, Artificial Sequence
  Description of Artificial Sequence Synthetic peptide
TFFYGGCMGNKNNFVREKY

SEQ ID NO: 40 - protein, 19 aa, Artificial Sequence
  Description of Artificial Sequence Synthetic peptide
TFFYGGSMGNKNNFVREKY

SEQ ID NO: 41 - protein, 19 aa, Artificial Sequence
  Description of Artificial Sequence Synthetic peptide
PFFYGGCLGNRNNYVREKY

SEQ ID NO: 42 - protein, 19 aa, Artificial Sequence
  Description of Artificial Sequence Synthetic peptide
TFFYGGCLGNRNNFVREKY

SEQ ID NO: 43 - protein, 19 aa, Artificial Sequence
  Description of Artificial Sequence Synthetic peptide
TFFYGGCLGNKNNYVREKY

SEQ ID NO: 44 - protein, 19 aa, Artificial Sequence
  Description of Artificial Sequence Synthetic peptide
TFFYGGCGGNGNNFLTAKY

SEQ ID NO: 45 - protein, 19 aa, Artificial Sequence
  Description of Artificial Sequence Synthetic peptide
TFFYGGCRGNRNNFLTAEY

SEQ ID NO: 46 - protein, 19 aa, Artificial Sequence
  Description of Artificial Sequence Synthetic peptide
TFFYGGCRGNGNNFKSAEY

SEQ ID NO: 47 - protein, 19 aa, Artificial Sequence
  Description of Artificial Sequence Synthetic peptide
PFFYGGCLGNKNNFKTAEY

SEQ ID NO: 48 - protein, 19 aa, Artificial Sequence
  Description of Artificial Sequence Synthetic peptide
TFFYGGCRGNRNNFKTEEY

SEQ ID NO: 49 - protein, 19 aa, Artificial Sequence
  Description of Artificial Sequence Synthetic peptide
TFFYGGCRGKRNNFKTEED

SEQ ID NO: 50 - protein, 19 aa, Artificial Sequence
  Description of Artificial Sequence Synthetic peptide
PFFYGGCGGNGNNFVREKY

SEQ ID NO: 51 - protein, 19 aa, Artificial Sequence
  Description of Artificial Sequence Synthetic peptide
SFFYGGCMGNGNNFVREKY

SEQ ID NO: 52 - protein, 19 aa, Artificial Sequence
  Description of Artificial Sequence Synthetic peptide
SFFYGGCMGNGNNFVREKY

SEQ ID NO: 53 - protein, 19 aa, Artificial Sequence
  Description of Artificial Sequence Synthetic peptide
TFFYGGCLGNGNNFVREKY

SEQ ID NO: 54 - protein, 19 aa, Artificial Sequence
  Description of Artificial Sequence Synthetic peptide
SFFYGGCLGNGNNYLREKY

SEQ ID NO: 55 - protein, 19 aa, Artificial Sequence
  Description of Artificial Sequence Synthetic peptide
TFFYGGSLGNGNNFVREKY

SEQ ID NO: 56 - protein, 19 aa, Artificial Sequence
  Description of Artificial Sequence Synthetic peptide
TFFYGGCRGNGNNFVTAEY

SEQ ID NO: 57 - protein, 19 aa, Artificial Sequence
  Description of Artificial Sequence Synthetic peptide
TFFYGGCLGKGNNFVSAEY

SEQ ID NO: 58 - protein, 19 aa, Artificial Sequence
  Description of Artificial Sequence Synthetic peptide
TFFYGGCLGNRNNFDRAEY

SEQ ID NO: 59 - protein, 19 aa, Artificial Sequence
  Description of Artificial Sequence Synthetic peptide
TFFYGGCLGNRNNFLREEY

SEQ ID NO: 60 - protein, 19 aa, Artificial Sequence
  Description of Artificial Sequence Synthetic peptide
TFFYGGCLGNKNNYLREEY

SEQ ID NO: 61 - protein, 19 aa, Artificial Sequence
  Description of Artificial Sequence Synthetic peptide
PFFYGGCGGNRNNYLREEY

SEQ ID NO: 62 - protein, 16 aa, Artificial Sequence
  Description of Artificial Sequence Synthetic peptide
PFFYGGSGGNRNNYLR

SEQ ID NO: 63 - protein, 19 aa, Artificial Sequence
  Description of Artificial Sequence Synthetic peptide
MRPDFCLEPPYTGPCVARI

SEQ ID NO: 64 - protein, 21 aa, Artificial Sequence
  Description of Artificial Sequence Synthetic peptide
ARIIRYFYNAKAGLCQTFVYG

SEQ ID NO: 65 - protein, 22 aa, Artificial Sequence
  Description of Artificial Sequence Synthetic peptide
YGGCRAKRNNYKSAEDCMRTCG

SEQ ID NO: 66 - protein, 22 aa, Artificial Sequence
  Description of Artificial Sequence Synthetic peptide
PDFCLEPPYTGPCVARIIRYFY

SEQ ID NO: 67 - protein, 19 aa, Artificial Sequence
  Description of Artificial Sequence Synthetic peptide
TFFYGGCRGKRNNFKTEEY

SEQ ID NO: 68 - protein, 19 aa, Artificial Sequence
  Description of Artificial Sequence Synthetic peptide
KFFYGGCRGKRNNFKTEEY

SEQ ID NO: 69 - protein, 19 aa, Artificial Sequence
  Description of Artificial Sequence Synthetic peptide
TFYYGGCRGKRNNYKTEEY

SEQ ID NO: 70 - protein, 19 aa, Artificial Sequence
  Description of Artificial Sequence Synthetic peptide
TFFYGGSRGKRNNFKTEEY

SEQ ID NO: 71 - protein, 20 aa, Artificial Sequence
  Description of Artificial Sequence Synthetic peptide
CTFFYGCCRGKRNNFKTEEY

SEQ ID NO: 72 - protein, 20 aa, Artificial Sequence
  Description of Artificial Sequence Synthetic peptide
TFFYGGCRGKRNNFKTEEYC

SEQ ID NO: 73 - protein, 20 aa, Artificial Sequence
  Description of Artificial Sequence Synthetic peptide
CTFFYGSCRGKRNNFKTEEY

SEQ ID NO: 74 - protein, 20 aa, Artificial Sequence
  Description of Artificial Sequence Synthetic peptide
TFFYGGSRGKRNNFKTEEYC

SEQ ID NO: 75 - protein, 19 aa, Artificial Sequence
  Description of Artificial Sequence Synthetic peptide
PFFYGGCRGKRNNFKTEEY

SEQ ID NO: 76 - protein, 19 aa, Artificial Sequence
  Description of Artificial Sequence Synthetic peptide
TFFYGGCRGKRNNFKTKEY

SEQ ID NO: 77 - protein, 19 aa, Artificial Sequence
  Description of Artificial Sequence Synthetic peptide
TFFYGGKRGKRNNFKTEEY

SEQ ID NO: 78 - protein, 19 aa, Artificial Sequence
  Description of Artificial Sequence Synthetic peptide
TFFYGGCRGKRNNFKTKRY

SEQ ID NO: 79 - protein, 19 aa, Artificial Sequence
  Description of Artificial Sequence Synthetic peptide
TFFYGGKRGKRNNFKTAEY

SEQ ID NO: 80 - protein, 19 aa, Artificial Sequence
  Description of Artificial Sequence Synthetic peptide
TFFYGGKRGKRNNFKTAGY

SEQ ID NO: 81 - protein, 19 aa, Artificial Sequence
  Description of Artificial Sequence Synthetic peptide
TFFYGGKRGKRNNFKREKY

SEQ ID NO: 82 - protein, 19 aa, Artificial Sequence
  Description of Artificial Sequence Synthetic peptide
TFFYGGKRGKRNNFKRAKY

SEQ ID NO: 83 - protein, 19 aa, Artificial Sequence
  Description of Artificial Sequence Synthetic peptide
TFFYGGCLGNRNNFKTEEY

SEQ ID NO: 84 - protein, 19 aa, Artificial Sequence
  Description of Artificial Sequence Synthetic peptide
TFFYGCGRGKRNNFKTEEY

SEQ ID NO: 85 - protein, 19 aa, Artificial Sequence
  Description of Artificial Sequence Synthetic peptide
TFFYGGRCGKRNNFKTEEY

SEQ ID NO: 86 - protein, 19 aa, Artificial Sequence
  Description of Artificial Sequence Synthetic peptide
TFFYGGCLGNGNNFDTEEE

SEQ ID NO: 87 - protein, 19 aa, Artificial Sequence
  Description of Artificial Sequence Synthetic peptide
TFQYGGCRGKRNNFKTEEY

SEQ ID NO: 88 - protein, 19 aa, Artificial Sequence
  Description of Artificial Sequence Synthetic peptide
YNKEFGTFNTKGCERGYRF

SEQ ID NO: 89 - protein, 19 aa, Artificial Sequence
  Description of Artificial Sequence Synthetic peptide
RFKYGGCLGNMNNFETLEE

SEQ ID NO: 90 - protein, 19 aa, Artificial Sequence
  Description of Artificial Sequence Synthetic peptide
RFKYGGCLGNKNNFLRLKY

SEQ ID NO: 91 - protein, 19 aa, Artificial Sequence
  Description of Artificial Sequence Synthetic peptide
RFKYGGCLGNKNNYLRLKY

SEQ ID NO: 92 - protein, 22 aa, Artificial Sequence
  Description of Artificial Sequence Synthetic peptide
KTKRKRKKQRVKIAYEEIFKNY

SEQ ID NO: 93 - protein, 15 aa, Artificial Sequence
  Description of Artificial Sequence Synthetic peptide
KTKRKRKKQRVKIAY

SEQ ID NO: 94 - protein, 17 aa, Artificial Sequence
  Description of Artificial Sequence Synthetic peptide
RGGRLSYSRRFSTSTGR

SEQ ID NO: 95 - protein, 10 aa, Artificial Sequence
  Description of Artificial Sequence Synthetic peptide
RRLSYSRRRF

SEQ ID NO: 96 - protein, 16 aa, Artificial Sequence
  Description of Artificial Sequence Synthetic peptide
RQIKIWFQNRRMKWKK

SEQ ID NO: 97 - protein, 19 aa, Artificial Sequence
  Description of Artificial Sequence Synthetic peptide
TFFYGGSRGKRNNFKTEEY

SEQ ID NO: 98 - protein, 58 aa, Artificial Sequence
  Description of Artificial Sequence Synthetic polypeptide
RPDFCLEPPYTGPCKARIIRYYFYNAKAGLCQTFVYGGCRAKRNNFKSAEDCMTCGGA

SEQ ID NO: 99 - protein, 19 aa, Artificial Sequence
  Description of Artificial Sequence Synthetic peptide
TFFYGGCRGKRNNFKTKEY

SEQ ID NO: 100 - protein, 19 aa, Artificial Sequence
  Description of Artificial Sequence Synthetic peptide
RFKYGGCLGNKNNYLRLKY

SEQ ID NO: 101 - protein, 19 aa, Artificial Sequence
  Description of Artificial Sequence Synthetic peptide
TFFYGGCRAKRNNFKRAKY

SEQ ID NO: 102 - protein, 35 aa, Artificial Sequence
  Description of Artificial Sequence Synthetic polypeptide
NAKAGLCQTFVYGGCLAKRNNFESAEDCMRTCGGA

SEQ ID NO: 103 - protein, 24 aa, Artificial Sequence
  Description of Artificial Sequence Synthetic peptide
YGGCRAKRNNFKSAEDCMRTCGGA

SEQ ID NO: 104 - protein, 22 aa, Artificial Sequence
  Description of Artificial Sequence Synthetic peptide
GLCQTFVYGGCRAKRNNFKSAE

SEQ ID NO: 105 - protein, 20 aa, Artificial Sequence
  Description of Artificial Sequence Synthetic peptide
LCQTFVYGGCEAKRNNFKSA

SEQ ID NO: 106 - protein, 6 aa, Artificial Sequence
  Description of Artificial Sequence Synthetic peptide
KRXXXK

SEQ ID NO: 107 - protein, 19 aa, Artificial Sequence
  Description of Artificial Sequence Synthetic peptide
TFFYGGSRGKRNNFKTEEY

SEQ ID NO: 108 - protein, 19 aa, Artificial Sequence
  Description of Artificial Sequence Synthetic peptide
RFFYGGSRGKRNNFKTEEY

SEQ ID NO: 109 - protein, 19 aa, Artificial Sequence
  Description of Artificial Sequence Synthetic peptide
RFFYGGSRGKRNNFKTEEY

SEQ ID NO: 110 - protein, 19 aa, Artificial Sequence
  Description of Artificial Sequence Synthetic peptide
RFFYGGSRGKRNNFRTEEY

SEQ ID NO: 111 - protein, 19 aa, Artificial Sequence
  Description of Artificial Sequence Synthetic peptide
TFFYGGSRGKRNNFRTEEY

SEQ ID NO: 112 - protein, 19 aa, Artificial Sequence
  Description of Artificial Sequence Synthetic peptide
TFFYGGSRGRRNNFRTEEY

SEQ ID NO: 113 - protein, 20 aa, Artificial Sequence
  Description of Artificial Sequence Synthetic peptide
CTFFYGGSRGKRNNFKTEEY

SEQ ID NO: 114 - protein, 20 aa, Artificial Sequence
  Description of Artificial Sequence Synthetic peptide
TFFYGGSRGKRNNFKTEEYC

SEQ ID NO: 115 - protein, 20 aa, Artificial Sequence
  Description of Artificial Sequence Synthetic peptide
CTFFYGGSRGRRNNFRTEEY

SEQ ID NO: 116 - protein, 20 aa, Artificial Sequence
  Description of Artificial Sequence Synthetic peptide
TFFYGGSRGRRNNFRTEEYC

SEQ ID NO: 117 - protein, 19 aa, Artificial Sequence
  Description of Artificial Sequence Synthetic peptide
TFFYGGXRGKRNNFKTEEY

SEQ ID NO: 118 - protein, 18 aa, Artificial Sequence
  Description of Artificial Sequence Synthetic peptide
FYGGXRGKRNNFKTEEYC

SEQ ID NO: 119 - protein, 16 aa, Artificial Sequence
  Description of Artificial Sequence Synthetic peptide
GGSRGKRNNFKTEEYC

SEQ ID NO: 120 - protein, 14 aa, Artificial Sequence
  Description of Artificial Sequence Synthetic peptide
SRGKRNNFKTEEYC

SEQ ID NO: 121 - protein, 12 aa, Artificial Sequence
  Description of Artificial Sequence Synthetic peptide
GKRNNFKTEEYC

SEQ ID NO: 122 - protein, 8 aa, Artificial Sequence
  Description of Artificial Sequence Synthetic peptide
KRNNFKYC

SEQ ID NO: 123 - protein, 6 aa, Artificial Sequence
  Description of Artificial Sequence Synthetic peptide
KRNNFK

SEQ ID NO: 124 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 451 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 58 aa, Artificial Sequence [sequence omitted]

SEQ ID NO: 127 - protein, 19 aa, Artificial Sequence
  Description of Artificial Sequence Synthetic peptide
YEETKFNNRKGRSGGYFFT

SEQ ID NO: 128 - protein, 19 aa, Artificial Sequence
  Description of Artificial Sequence Synthetic peptide
XFXYGGXXXKXNNXKXXXX

SEQ ID NO: 129 - protein, 30 aa, Artificial Sequence
  Description of Artificial Sequence Synthetic polypeptide
GGGGSGGGGSGGGGSGGGGSGGGGSGGGGS

SEQ ID NO: 130 - protein, 25 aa, Artificial Sequence
  Description of Artificial Sequence Synthetic peptide
XXXXGXXXXGXXXXGXXXXGXXXXG

SEQ ID NO: 131 - protein, 5 aa, Artificial Sequence
  Description of Artificial Sequence Synthetic peptide
SSSSG

SEQ ID NO: 132 - protein, 5 aa, Artificial Sequence
  Description of Artificial Sequence Synthetic peptide
PAPAP

SEQ ID NO: 133 - protein, 15 aa, Artificial Sequence
  Description of Artificial Sequence Synthetic peptide
PTPTPTPTPTPTPTP

SEQ ID NO: 134 - protein, 27 aa, Artificial Sequence
  Description of Artificial Sequence Synthetic peptide
AEAAAKEAAAKEAAAKEAAAKEAAAKA

SEQ ID NO: 135 - protein, 4 aa, Artificial Sequence
  Description of Artificial Sequence Synthetic peptide
ALAL

SEQ ID NO: 136 - protein, 4 aa, Artificial Sequence
  Description of Artificial Sequence Synthetic peptide
GFLG

SEQ ID NO: 137 - protein, 58 aa, Artificial Sequence [sequence omitted]

SEQ ID NO: 138 - protein, 20 aa, Artificial Sequence
  Description of Artificial Sequence Synthetic peptide
CTFFYGGSRGKRNNFKTEEY

SEQ ID NO: 139 - protein, 19 aa, Artificial Sequence
  Description of Artificial Sequence Synthetic peptide
TFFYGGSRGKRNNFKTEEY

SEQ ID NO: 140 - protein, 6 aa, Artificial Sequence
  Description of Artificial Sequence Synthetic peptide
KRXXXK

(19) United States
(12) Patent Application Publication
(10) Pub. No.: US 2016/0106856 A1
(43) Pub. Date: Apr. 21, 2016

(54) CONJUGATES INCLUDING AN ANTIBODY MOIETY, A POLYPEPTIDE THAT TRAVERSES THE BLOOD-BRAIN BARRIER, AND A CYTOTOXIN

(71) Applicant: AngioChem Inc., Montreal (CA)

(72) Inventors: Jean-Paul Castaigne, Mont-Royal (CA); Gaoqiang Yang, Montreal (CA); Christian Che, Longueuil (CA); Michel Demeule, Beaconsfield (CA)

(21) Appl. No.: 14/421,213

(22) PCT Filed: Aug. 14, 2013
(86) PCT No.: PCT/CA2013/050625

§ 371 Date: Feb. 12, 2015

Related U.S. Application Data
(60) Provisional application No. 61/865,071, filed on Aug. 12, 2013.
(60) Provisional application No. 61/682,991, filed on Aug. 14, 2012.

(51) Int. Cl. A61K 47/48; C07K 16/30; C07K 16/28; C07K 16/32; A61K 31/337

(52) U.S. Cl. CPC A61K 47/48246; A61K 47/48561; A61K 47/48584; C07K 16/32; A61K 47/48384; A61K 31/337; C07K 16/2863; C07K 16/3015; C07K 2317/24

(57) ABSTRACT

The present invention relates to antibody-polypeptide-cytotoxin conjugates and methods of making, packaging, and using the conjugates. The polypeptide can be a Kunitz-type protease inhibitor or a derivative thereof that facilitates transport of the conjugate across the blood-brain barrier and/or into cancer cells outside the CNS, and the antibody moiety selectively binds a target within the CNS or in peripheral tumors to direct the cytotoxic agent to that target (e.g., a tumor or cancer cell). The conjugates can be further defined by the inclusion of a linker between the antibody moiety and the polypeptide; by the number of polypeptides and cytotoxic agents conjugated thereto; by the positions at which the entities within the conjugates are bound to one another; and by the larger configuration of the conjugate. Modified polypeptides (e.g., polypeptides conjugated to cytotoxic agents but not to an antibody moiety), pharmaceutical compositions, kits (e.g., including a modified polypeptide and an as-yet unconjugated antibody), and methods of making and using the conjugates are also features of the invention.

### CROSS REFERENCE TO RELATED APPLICATIONS

This application claims the benefit of the filing date of U.S. Application No. 61/682,991, which was filed on Aug. 14, 2012, and the benefit of the filing date of U.S. Application No. 61/865,071, which was filed on Aug. 12, 2013. For the purpose of any later-filed U.S. utility application(s) and/or U.S. patent(s) that claim priority to one or both of these provisional applications, the content of the provisional applications is hereby incorporated by reference in its entirety.

### FIELD OF THE INVENTION

The present invention relates to protein conjugates in which one or more Kunitz-type protease inhibitors (e.g., aprotinin) or derivatives thereof (e.g., an aprotinin-derived polypeptide) are conjugated to an antibody moiety and to a cytotoxic agent in the manner described herein; methods by which the conjugates are synthesized for use; physiologically acceptable compositions including them; and methods of administering the conjugates to patients for the treatment of cancer as described herein.

### BACKGROUND OF THE INVENTION

The brain is protected from exposure to potentially toxic, ingested substances by two barrier systems: the blood-brain barrier (BBB) and the blood-cerebrospinal fluid barrier (BCSFB). While theses barriers protect the central nervous system (CNS) from harmful substances (e.g., accidentally ingested toxins), they also prevent therapeutic proteins from accessing the brain and spinal cord and, therefore, present major obstacles in treating disorders of the CNS. As a general rule, only lipophilic molecules smaller than about 500 Daltons traverse the BBB. Many drugs that show promising results in animal studies of CNS disease are considerably larger than that, and protein therapeutics are generally excluded from the CNS altogether due to the negligible permeability of the brain capillary endothelial wall to drugs of that size and complexity. Treating patients with brain cancers, whether neuromas or gliomas, has been particularly challenging. With some malignancies, most patients survive for less than one year despite surgical resection, radiation therapy and/or systemic chemotherapy.

The strategies currently being pursued to enhance delivery of protein therapeutics to the CNS can generally be divided into three categories. The first category includes invasive procedures such as direct intraventricular administration of drugs and intra-carotid infusion of hyperosmolar solutions that temporarily disrupt the BBB. The second category includes pharmacologically-based strategies that are aimed at increasing the lipid solubility of proteins through the BBB. In the third category are delivery regimes in which the therapeutic agent is attached to a protein that acts as a vector or receptor-targeted delivery vehicle with respect to the BBB. This third approach is advantageous in that it is highly specific and efficacious, results in minimal untoward effects, and is broadly applicable.

Outside the CNS, the targeted delivery of therapeutic agents to only diseased cells remains a pervasive challenge. Systemic chemotherapy is effective in treating some kinds of cancers, but with many others it fails because the doses required to achieve control of tumor growth are frequently so high that they cause unacceptable systemic toxicity.

### SUMMARY OF THE INVENTION

In a first aspect, the present invention features protein conjugates that include an antibody moiety, one or more Kunitz-type protease inhibitors (e.g., aprotinin) or derivatives thereof (e.g., an aprotinin-derived polypeptide), and a cytotoxic agent. The protein conjugates can be further defined by the inclusion of a linker between the antibody moiety and the polypeptide; the inclusion of a linker between the polypeptide and the cytotoxic agent; and by the configuration of the antibody moiety, polypeptide, and cytotoxic agent within the conjugate. Thus, in one embodiment, the invention features a protein conjugate that includes an antibody moiety, a polypeptide, and a cytotoxic agent, with the polypeptide including the amino acid sequence Lys-Arg-Asn-Asn-Phe-Lys (SEQ ID NO:123) or a biologically active analog thereof. Generally, the conjugates can be essentially linear (insofar as the tertiary structure of the component proteins allows) or branched. As described further below, linear configurations are achieved by joining the component parts of the conjugate to one another directly or with bifunctional linkers, and branched configurations are achieved by including at least one linker that has three or more functional substituents (e.g., a trifunctional linker that links one antibody moiety with two polypeptides or a tetrafunctional linker that links one antibody and three polypeptides, for example). Thus, the conjugates described herein can include a linker between the antibody moiety and the polypeptide and/or between the polypeptide and the cytotoxic agent. Where the linker is a homofunctional linker, it can be a homobifunctional, homotrifunctional, or homotetrafunctional linker that includes two, three, or four reactive groups, respectively, and these reactive groups (or substituents) can react with a primary amine, a thiol group, a hydroxyl group, or a carbohydrate. Where the linker is a heterofunctional linker, it can be a heterobifunctional, heterotrifunctional, or heterotetrafunctional linker that includes at least one reactive group (also known as a substituent) that reacts with a primary amine, a thiol group, a hydroxyl group, or a carbohydrate. Linkers with more than four reactive groups (e.g., 5-10 reactive groups) can also be used. More specifically, a protein conjugate can include, as the linker, a monofluoro cyclooctyne (MFCO), bicyclo[6.1.0]nonyne (BCN), dibenzocyclooctyne (DBCO), N-succinimidyl-S-acetylthioacetate (SATA), N-succinimidyl-S-acetylthiopropionate (SATP), or N-Hydroxy-succinimide (NHS).

While polypeptides are described further below, we note here that the protein conjugates of the invention can include a polypeptide that includes the amino acid sequence Thr₁-Phe₂-Phe₃-Tyr₄-Gly₅-Gly₆-Cys₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉ (SEQ ID NO:67) or an analog thereof or Thr₁-Phe₂-Phe₃-Tyr₄-Gly₅-Gly₆-Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉ (SEQ ID NO:97) or an analog thereof. Unless the context clearly indicates otherwise, a composition described herein, such as a polypeptide, that includes a component part (e.g., a specified sequence) can include only the component part (e.g., only the specified sequence in the case of a polypeptide) or the component part and more (e.g., the specified sequence with additional residues at either or both termini in the case of a polypeptide). Where the polypeptide is an analog of a particular sequence described herein (the reference polypeptide), one or more of the cysteine, serine, and lysine residues in the reference polypeptide can remain invariant. For example, where the conjugate includes an analog of SEQ ID NO:67, at least 13 amino acid residues, including Cys₇, Lys₁₀, and Lys₁₅ can remain invariant; where the conjugate includes an analog of SEQ ID NO:97, at least 13 amino acid residues, including Ser₇, Lys₁₀, and Lys₁₅, can remain invariant. In an analog of SEQ ID NO:67 or SEQ ID NO:97, Asn₁₂ can be substituted with Gln, Asn₁₃ can be substituted with Gln, and/or Phe₁₄ can be substituted with Tyr or Trp. The analog can include, for example, the sequence Phe₃-Tyr₄-Gly₅-Gly₆-Cys₇/Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:118); Gly₅-Gly₆-Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:119); Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:120); Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:121); or Lys₁₀-Arg₁₁-Asn₁₂-Asn₁₃-Phe₁₄-Lys₁₅-Tyr₁₉-Cys (SEQ ID NO:122). In any of the polypeptides, at least one amino acid residue can be present in the D-form.

With regard to the number of component parts, a conjugate can include many more polypeptides than antibody moieties (see the description of possible configurations below). In some embodiments, the conjugates can include 1-10 polypeptides and one antibody moiety, and the ratio of polypeptides to antibody moiety can be 1:1-10:1 (other ratios are possible in accordance with the description below). In some embodiments, the conjugate includes 1-6 polypeptides. The polypeptide and the cytotoxic agent can be present in a ratio of 1:1 to 1:3 (polypeptide: cytotoxic agent).

Each polypeptide can be linked, via at least one linker, to an antibody moiety, and the antibody moiety can be a tetrameric antibody or a biologically active variant thereof. For example, the antibody moiety can be a single chain antibody (scFv), Fab fragment, or F(ab′)2 fragment; can be a human, chimeric or humanized antibody or a biologically active variant thereof; and/or can be (or can be derived from) a monoclonal or polyclonal antibody. With regard to the target to which the antibody moiety specifically binds, the target can be a growth factor receptor or an interleukin receptor. For example, the growth factor receptor can be a member of the epidermal growth family receptor (EGFR) family, and the antibody moiety can trastuzumab, cetuximab, or panitumumab, or a biologically active variant thereof. In other embodiments, the growth factor receptor can be a vascular endothelial growth factor receptor (VEGFR). In other embodiments, the interleukin receptor can be an IL-2 receptor (in which case the antibody moiety can be, for example, basiliximab, daclizumab, or a biologically active variant thereof) or an IL-6 receptor (in which case the antibody moiety can be, for example, tocilizumab or a biologically active variant thereof). In other embodiments, the growth factor receptor is a TNF-α receptor. More generally, the antibody moiety can be an anti-cancer agent or an anti-inflammatory agent.

The cytotoxic agent can be a taxane (e.g., docetaxel or an active variant thereof), an alkaloid (e.g., a vinca alkaloid), an anthracycline (e.g., doxorubicin), an auristatin (e.g., monomethyl auristatin E (MMAE)), an antifolate (e.g., methotrexate or aminopterin), a calicheamicin (e.g., calicheamicin γ 1), a duocarmycin (e.g., adozelesin, bizelesin, or carzelesin), a mitomycin (e.g., mitomycin C); a pyrimidine analog (e.g., fluorouracil); or a derivative of mytansine (e.g., a mytansinoid such as ansamitocin, mertansine, or emtansine).

As noted, the antibody moiety, the polypeptide, and the cytotoxic agent can be linked in a linear conjugate or configured as a dendrimeric conjugate.

In another aspect, the invention features pharmaceutical compositions that include a conjugate as described herein and a pharmaceutically acceptable carrier. These compositions can be formulated for intravenous administration.

In another aspect, the invention features methods of treating a patient who is suffering from cancer. The method can include the step of identifying a patient in need of treatment (e.g., a human patient who has a primary or secondary tumor (e.g., a tumor within the patient's brain or spinal cord)), and includes the step of administering to the patient a therapeutically effective amount of a pharmaceutical composition that includes a protein conjugate as described herein. The patient's cancer can be associated with expression of HER-2 (e.g., a breast, ovarian, lung, or gastric cancer). The patient's cancer can be associated with the expression of an epidermal growth factor receptor (e.g., a head and neck cancer or colon cancer).

The present invention also features methods of producing the conjugates described herein; methods of producing pharmaceutical compositions that include them; and the use of these conjugates and compositions in the treatment of disease, including cancer, inflammation, and the specific cancers described herein. Any of the methods of treatment can be configured as methods of “use”. Accordingly, the invention features the use of a protein conjugate as described herein in the preparation of a medicament and the use of a protein conjugate as described herein the preparation of a medicament for the treatment of a disease or disorder (including cancer, inflammation, and the specific cancers described herein).

We use the term “protein conjugate” to refer to an amino acid-based compound formed of molecularly coupled (e.g., covalently bonded) parts. In the present invention, the parts include an antibody moiety that specifically binds a selected target, a Kunitz-type protease inhibitor (e.g., aprotinin) or a derivative thereof (e.g., an aprotinin-derived polypeptide) that facilitates the transport of the conjugate across the blood-brain barrier and/or into targeted cells, and a cytotoxic agent. For ease of reading, we will refer to the Kunitz-type protease inhibitor or a derivative thereof as a “polypeptide”. An “amino acid-based compound” is one that includes primarily, but not necessarily exclusively, amino acid residues. As noted above, the protein conjugates can include a linker, which may be a chemical compound (or otherwise recognized as a chemical entity as opposed to an amino acid or protein). The cytotoxic agent can also be a chemical compound. Such conjugates would include primarily, but not necessarily exclusively, amino acid residues, as the antibody moiety and polypeptide would be made of amino acids while the linker and cytotoxic agents would be chemical entities. As described further below, in some embodiments, the cytotoxin can also be a protein, increasing the proportion of the conjugate that is formed from amino acids. The protein conjugates may also include a detection agent, which may or may not be a protein. For example, the detection agent can be a fluorophor, fluorescent protein, radioisotope, dye, or the like. The invention encompasses methods in which the detection agent is detected to, for example, analyze the delivery of the conjugate to a tumor or cancer cells within the CNS or within another tissue or cell type external to the CNS.

For the sake of added clarity, we note that the antibody moieties and the polypeptides are distinct from one another. Further, any protein conjugate can include more than one polypeptide, and the two, three, four, or more polypeptides may be the same (i.e., may have an identical amino acid sequence) or may differ from one another (i.e., the polypeptides conjugated to the antibody moiety may have different amino acid sequences). With respect to the compositions and methods of the invention, we use the term “include(s)” to mean “comprising.” In any instance, unless the context specifically indicates otherwise, the compositions of the invention, their component parts (e.g., the antibody moiety and the polypeptide), and the methods of the invention can either comprise or consist of the recited elements or steps. For example, in any embodiment, a given polypeptide can comprise or consist of the recited sequence (e.g., SEQ ID NO:67, 97, 117 or 123).

As indicated above, the protein conjugates of the present invention can include a polypeptide as described herein or a biologically active analog or fragment thereof. We use the term “biologically active” with respect to a given polypeptide to mean an analog or fragment of that polypeptide that retains sufficient activity (e.g., receptor binding activity) in a physiological setting to be useful. For example, where a given polypeptide facilitates the transport of protein conjugate of which it is a part across the BBB, an analog or fragment of that polypeptide is biologically active when it also has the ability, under the same or comparable conditions, to transport the conjugate across the BBB. Any such property (e.g., receptor binding, cellular internalization, or BBB traversal) can be tested in vivo or in ex vivo models, and biologically active analogs and fragments of a referenced polypeptide may be more or less effective than the referenced polypeptide. In particular, the efficacy of a fragment or analog may be lower than that of the referenced polypeptide as long as it remains high enough to achieve a desired outcome (e.g., as long as the conjugate in which it is included achieves a clinically beneficial result).

A “fragment” of a referenced polypeptide is a continuous or contiguous portion of the referenced polypeptide (e.g., a fragment of a polypeptide that is ten amino acids long can be any 2-9 contiguous residues within that polypeptide). An “analog” of a referenced polypeptide is any polypeptide having a sequence that is similar to, but not identical to, the sequence of the referenced polypeptide or a portion thereof. Thus, a polypeptide that includes one or more amino acid substitutions, additions, or deletions of an amino acid residue (or any combination thereof) is an analog of the referenced sequence, and a fragment is a type of analog. Fragments and analogs of polypeptides suitable for inclusion in the present protein conjugates are further described below.

In addition to BBB permeability, the protein conjugates of the invention may have one or more of the following advantages: little or no precipitation at dosing concentrations; freeze-thaw stability; and high solubility (producing few if any aggregates in solution). The antibody moiety can also retain high affinity for its target (relative to the affinity in an unconjugated form). For example, the antibody moiety can have an affinity for its target that is within about 3-fold of the affinity of the moiety when unconjugated. In studies with the protein conjugate H43 (comprising an angiopep-2 polypeptide and an antibody that specifically binds HER2) we observed an affinity of 2.2 nM where the affinity of the unconjugated antibody moiety was 1.3 nM. The inclusion of a cytotoxin is particularly advantageous in the event a cancerous cell becomes refractory to antibody treatment. Other features and advantages of the present compositions and methods are illustrated in the description below, the drawings, and the claims.

### BRIEF DESCRIPTION OF THE DRAWINGS

FIG. 1 is a Table showing representative polypeptides that can be incorporated into the present protein conjugates.

FIGS. 2a-2d are illustrations of four ways in which an antibody moiety (represented on the left by a gray oval) can be conjugated to a polypeptide that has, in turn, been conjugated to a cytotoxic agent. In FIG. 2(a) (uppermost), the conjugation is achieved by click chemistry; a cyclooctyne linked to the antibody moiety reacts with the azide group at the N-terminus of the polypeptide. (The two reactive substituents could be reversed, with the cyclooctyne linked to the polypeptide and the antibody modified to include an azide.) In FIGS. 2(b)-2(d), the antibody is joined to the polypeptide via a reactive thiol group. In FIG. 2(b), the polypeptide bears a thiol group (C—SH) at the N-terminus, which can react with a maleimide-containing linker such as Mal-AMCHC-OSu (trans-N-Succinimidyl 4-(maleimidomethyl)cyclohexane-1-carboxylate; a heterobifunctional cross-linking reagent with amine and thiol reactivity) or SPDP (N-succinidmidyl 3-[2-pyridyldithio]-propionate). While the polypeptide portion of the protein conjugate can be modified to include a thiol group, as shown in FIG. 2(b), the conjugates can be formed instead by reacting a thiol-bearing antibody moiety with a thiol-reactive linker bound to the polypeptide. In FIG. 2(c), a pyridinyl disulfide group extending from the polypeptide reacts with a thiol-bearing antibody moiety. In FIG. 2(d), a maleimide-containing linker extending from the polypeptide reacts with a thiol-bearing antibody moiety. The cytotoxic agent is conjugated to the lysine residues within the polypeptide via an aliphatic linker, with an amide bond to polypeptide and an ester bond to cytotoxic agent.

FIG. 3 illustrates a conjugation scheme in which a single cytotoxic agent is conjugated via a thiol-reactive linker to a thiol-modified C-terminus of a representative polypeptide. While the antibody is linked to a cyclooctyne in anticipation of a reaction with the azide-containing N-terminus of the polypeptide, the antibody and the polypeptide can be linked in any manner described herein, including those illustrated in FIGS. 2(b)-2(d).

FIGS. 4(a)-4(c) illustrate three ways in which the present conjugates can be configured in dendrimeric form. As in FIG. 2(a), the antibody and polypeptide are configured to be joined by a click reaction. A single branch comprising an alkyl (C₁-C₆ alkyl) extends from the antibody moiety to a first branch moiety that serves as the core (X_{core} in Formula I) for the branches that extend therefrom. In FIGS. 4(a)-4(c), the branched portions of the conjugates have four branches, and all four of these may terminate in a polypeptide that is linked to two copies of a cytotoxic agent (as in 4(b), where the polypeptides are linked to the conjugate by a pyridinyl disulfide bond, and 4(c), where the polypeptides are linked to the conjugate by maleimido-hexanoic acid). Alternatively, two branches of the four branches can terminate in a polypeptide and the remaining two can terminate with a cytotoxic agent (as in 4(a)).

FIGS. 5(a)-5(c) illustrate three further ways the present conjugates can be configured in dendrimeric form. In FIGS. 5(a) and 5(b), an antibody moiety is linked to a dendrimer core moiety from which two branches extend. At the end of each branch is a polypeptide-linked cytotoxin. In FIG. 5(c), four branches extend from a central core, with the terminus of one branch being linked to an antibody moiety and the termini of the remaining three branches being linked to a polypeptide-linked cytotoxin.

FIG. 6 is a scheme for conjugating a drug to modified polypeptide (N₃An2-(SuDoce)₂).

FIG. 7 is a line graph illustrating the results of a cytotoxicity assay carried out in BT-474 cells. The cells were treated for five days with ANG 4043 and YG-51-42-A1. The former is a conjugate including an antibody that binds HER2 and the polypeptide represented by SEQ ID NO:97 (Angiopep-2 (An2)). The latter is a conjugate including that same antibody and aprotinin-derived polypeptide as well as the cytotoxic agent docetaxel. While the IC₅₀ for ANG4043 was 2.228 nM, the IC₅₀ for YG-51-42-A1 was only 0.297 nM.

FIGS. 8a-8h illustrate portions of conjugates that include branching structures that can be included in the present conjugates. The conjugates can include the polypeptides as illustrated in this drawing or another polypeptide as described herein together with one or more of the antibody moieties and cytotoxins described herein.

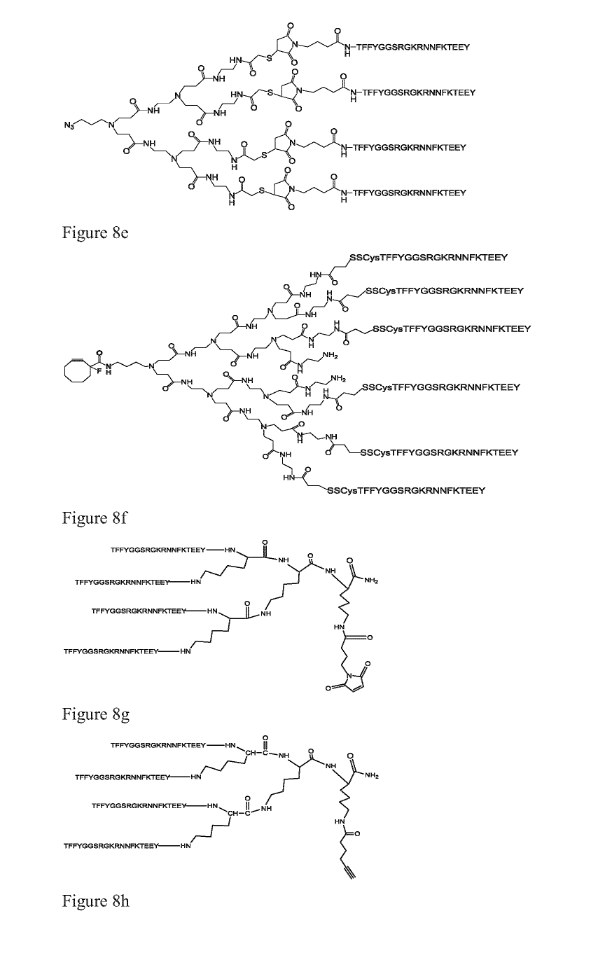

### DETAILED DESCRIPTION OF THE INVENTION

The majority of the drugs that are potentially useful in treating the CNS do not cross the BBB, and it is surprising that so little effort has been focused on this longstanding problem. It is estimated that more than 99% of CNS drug development worldwide is devoted to drug discovery per se, with less than 1% of the effort directed to improving delivery. There are no reliably effective treatments for many serious, costly, and debilitating CNS disorders, including brain cancers. Thus, there is a substantial and unmet need for not only discovering effective agents, but also for successfully delivering those agents to the brain and spinal cord. Therapeutic antibodies are among the most promising new treatments for many types of cancers. However, due to their size, therapeutic antibodies are among the most difficult agents to deliver to the CNS. Our studies have demonstrated that antibodies conjugated to an aprotinin-derived polypeptide can be delivered to the CNS more effectively and, further, that the therapeutic benefit of such conjugates can be enhanced for patients affected by cancer by the inclusion of a cytotoxin. In the context of the present invention, a conjugate comprising an antibody moiety, a polypeptide, and a cytotoxic agent crosses the BBB to a greater extent than the antibody moiety would have crossed the blood-brain barrier alone (i.e., without conjugation to the polypeptide). Differences in transport can also be observed in ex vivo models of the BBB. Thus, the polypeptides in the present conjugates are useful in transporting the antibody moiety and a cytotoxic agent across the blood-brain barrier of an individual, and the resulting, improved access to the CNS allows the antibody moiety and the cytotoxic agent to be used in the treatment of CNS cancers in ways not previously possible. As also described herein, the present conjugates are also useful in treating cancers in which either a primary or secondary tumor develops outside the CNS.

The present invention extends the inventors previous work with physiologic-based strategies for drug delivery. Here, the conjugates further include a cytotoxic agent that provides additional therapeutic benefit. Although many antibodies have demonstrated selectivity for tumor or cancer cells, their clinical use is limited because of poor therapeutic efficacy in human patients. Likewise, known cytotoxins for the treatment of cancer have limited use clinically because of their toxicity profiles. The present conjugates improve therapeutic drug delivery and treatment of cancers in the CNS and elsewhere within acceptable safety parameters. Thus, an advantage of the present conjugates is their combined ability to cross the BBB, preferentially target tumor and/or cancer cells in both the CNS and other tissues and ultimately deliver therapeutically effective concentrations of a cytotoxic agent to those cells. The protein conjugates described herein are capable of not only crossing the blood-brain barrier (BBB), but also of delivering the cytotoxin they bear to particular peripheral cell types, including cells in the breast, colon, liver, lung, spleen, kidney, ovaries, and muscle, with enhanced efficiency. More generally, the present conjugates are capable of targeting any cell or tissue that expresses a low-density lipoprotein receptor-related protein (LRP1). This receptor is a member of the LDL-receptor family, which also includes LRP2 (also known as megalin), and it mediates the transport of ligands across endothelial cells of the BBB (Shibata et al., 2000; Ito et al., 2006; Bell et al., 2007). More specifically, LRP2 acts as a multi-ligand binding receptor at the plasma membrane of epithelial cells and mediates endocytosis of ligands leading to transcytosis. In the following paragraphs, we first describe the component parts of the present protein conjugates, then the manner in which they can be configured, and the linkers useful in such configurations.

Polypeptides:

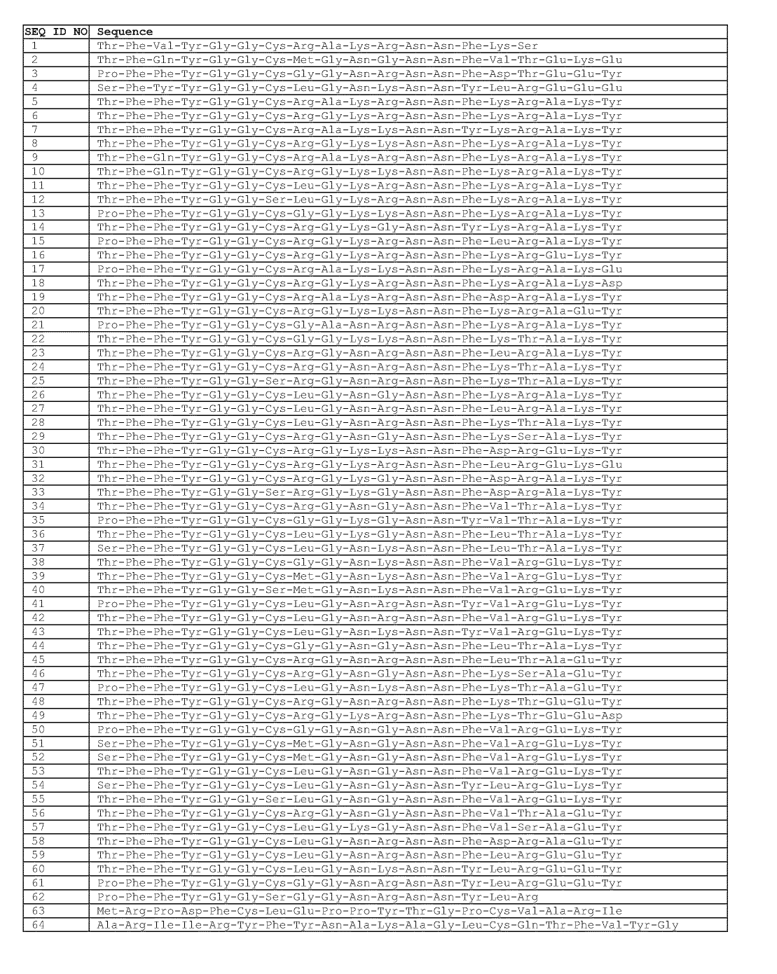

Aprotinin is a protease inhibitor of the Kunitz-type (i.e., it contains a KPI domain). It is a ligand for LRP1 and LRP2, and in vitro studies have demonstrated that aprotinin crosses a cell layer mimicking the mammalian BBB. Although the exact molecular mechanism of transcytosis is unclear, and while the invention is not limited to protein conjugates that function by any particular molecular mechanism, the polypeptides described herein, including aprotinin and aprotinin-derived polypeptides, are thought to interact with a receptor in the LDL receptor family. The inventors have identified polypeptides that differ from, but retain some degree of structural and functional similarity to, an aprotinin polypeptide. For example, the inventors have identified both 19-amino acid polypeptides and, within those, 6-amino acid polypeptides that are useful within the present conjugates. The 19-amino acid polypeptides correspond to residues 32-50 of aprotinin (SEQ ID NO:126) and conform to the sequence Xaa₁-Phe-Xaa₃-Tyr-Gly-Gly-Xaa₇-Xaa₈-Xaa₉-Lys-Xaa₁₁-Asn-Asn-Xaa₁₄-Lys-Xaa₁₆-Xaa₁₇-Xaa₁₈-Xaa₁₉ (SEQ ID NO:128), where Xaa₁ is Thr, Pro, or Ser; Xaa₃ is Val, Gln, Phe, or Tyr; Xaa₁₄ is Cys or Ser; Xaa₈ is Arg, Met, Gly, or Leu; Xaa₉ Gly or Ala; Xaa₁₁ is Gly, Arg, or Lys; Xaa₁₄ is Phe or Tyr; Xaa₁₆ is Thr, Arg, or Ser; Xaa₁₇ is Gly or Ala; Xaa₁₈ is Lys or Glu; and Xaa₁₉ Gly, Tyr, or Asp. SEQ ID NOs: 1-61 of FIG. 1 conform to SEQ ID NO:126. The 6-amino acid polypeptides correspond to residues 41-46 of aprotinin (SEQ ID NO:126) and conform to the sequence Lys-Arg-Xaa₃-Xaa₄-Xaa₅-Lys (SEQ ID NO:106), where Xaa₃ is Asn, Ser, Thr, or Gln; Xaa₄ is Asn, Ser, Thr, or Gln; and Xaa₅ is Phe or Tyr. For example, a conjugate as described herein can include a polypeptide including the sequence Lys-Arg-Asn-Asn-Phe-Lys (SEQ ID NO:123).

A polypeptide incorporated in the present conjugates can include or consist of about 6-60 amino acid residues, and useful polypeptides can include or consist of a sequence conforming to SEQ ID NO:106 or 128. Any given polypeptide can exhibit a degree of homology or identity to an aprotinin sequence (e.g., 70-100% homology or identity); include about ten lysine and/or arginine residues; contain no more than about four negatively-charged residues (e.g., no more than about four aspartate or glutamate residues); include about three (e.g., 2-4, inclusive) intramolecular (e.g., disulfide) bonds; and/or include a twisted β-hairpin and/or C-terminal a helix. Among the longer useful polypeptides are SEQ ID NOs 98 and 126.

The polypeptides within the conjugates may have (consist of) or include (comprise) a sequence as specifically set out herein (e.g., a sequence represented by SEQ ID NO:67 or 97 or any other of the aprotinin-derived polypeptides shown in the Table of FIG. 1) or they may be biologically active fragments or analogs of any of these reference sequences. A fragment differs from a reference sequence by virtue of having fewer contiguous amino acid residues (one or more amino acids from the N- or C-terminal are deleted) whereas an analog differs from the reference sequence by virtue of including at least one additional amino acid residue or at least one amino acid substitution. The additional amino acid residue(s) can be added to the N-terminal, the C-terminal, to a position between the termini of a reference polypeptide (e.g., SEQ ID NO:117), or at any combination of these positions. An analog may be shorter than a reference sequence (i.e., it may include a deletion of one or more amino acid residues), however, we use the term “analog” to mean a variant that differs in some way from the reference sequence other than just a simple deletion of an N- and/or C-terminal amino acid residue or residues. Where the analog includes a substitution of an amino acid residue, the substitution may be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

Where a reference sequence has 19 amino acid residues (e.g., a polypeptide conforming to SEQ ID NO:126), a biologically active fragment thereof may have 6-18 contiguous residues (inclusive), and a biologically active analog thereof may have 1-13 amino acid substitutions; one or more amino acid additions (e.g., the addition of residues at the N- and/or the C-terminal that increase the length of the reference polypeptide to up to about 50-60 amino acid residues); or a combination of such substitutions and additions. As noted, in the case of an analog (as opposed to a fragment), where there is at least one substitution and/or at least one addition, there may also be at least one deletion.

The degree of similarity between a first polypeptide (e.g., a reference polypeptide such as aprotinin or another polypeptide listed in FIG. 1) and a second polypeptide may be expressed as the percentage of the residues that are identical at comparable positions. Polypeptides useful in the present conjugates can be at least 35%, 40%, 50%, 60%, 70%, 80%, 85%, 90%, 95%, or 98% identical to a reference polypeptide. For example, a polypeptide within a protein conjugate can have an amino acid sequence that is at least 80% (e.g., at least 85%, 90%, 95%, or 98%) identical to a sequence selected from the group consisting of SEQ ID NOs:1-105 and 107-123. Our studies to date suggest that a preferred polypeptide will have or include the amino acid sequence of Angiopep-1 (SEQ ID NO:67), Angiopep-2 (An2) (SEQ ID NO:97), Cys-Angiopep-2 (CysAn2) (SEQ ID NO:113), Angiopep-2-Cys (SEQ ID NO:114), or reversed Angiopep-2 (SEQ ID NO:127).

The amino acid residues within the polypeptides can be of the standard α-amino acid form and can be in the D-form, the L-form, or a mixture of these two enantiomeric forms. As is known in the art, all α-amino acids except glycine can exist in either of two enantiomeric forms, and either or both forms can be incorporated in the present polypeptides as well as in the antibody moieties of the protein conjugates. Substituting a D-form amino acid residue in the place of an L-form amino acid residue may generate a more stable polypeptide. For example, employing D-lysine in place of L-lysine at position 10 and/or position 15 (or comparable positions in an analog of an aprotinin-derived polypeptide) may increase the stability of an aprotinin-derived polypeptide, and the use of all such D-lysine-containing polypeptides is within the scope of the present invention. Similarly, employing a D-form amino acid at the N-terminal and/or C-terminal of such a polypeptide should increase in vivo stability because peptidases cannot utilize a D-amino acid as a substrate (Powell et al., Pharm. Res. 10:1268-1273, 1993). The polypeptides can also be configured as reverse-D polypeptides, which contain D-form amino acid residues arranged in a reverse sequence relative to a polypeptide containing L-amino acids; the C-terminal residue of an L-amino acid polypeptide becomes N-terminal for the D-amino acid polypeptide, and vice versa. Reverse D-polypeptides retain the same tertiary conformation and therefore the same activity as the corresponding L-amino acid polypeptide, but their increased stability can lead to greater therapeutic efficacy (Brady and Dodson, Nature 368:692-693, 1994 and Jameson et al., Nature 368:744-746, 1994). The polypeptides may also be “constrained” by, for example, the addition of cysteine residues that form disulfide bridges (see Rizo et al., Ann. Rev. Biochem. 61:387-418, 1992). In any embodiment, the polypeptide can be a cyclic polypeptide.

With respect to their preparation, polypeptides useful in protein conjugates can be produced by any methods known in the art, including by synthetic methods and recombinant techniques used routinely to produce proteins from nucleic acids. The resulting polypeptides may be stored in an unpurified or in an isolated or substantially purified form until further use. For example, the polypeptides can be stored until used in the methods described below for generating a protein conjugate of the invention. Chemical synthesis can be achieved, for example, by solid phase synthesis, and recombinant techniques can include expression from vector constructs in a biological cell (e.g., a prokaryotic or eukaryotic cell). Codons that encode specific amino acid residues are well known in the art (see, e.g., “Biochemistry,” 3rd Ed., 1988, Lubert Stryer, Stanford University, W.H. Freeman and Company, New-York), as are methods for codon optimization. An exemplary nucleotide sequence encoding an aprotinin analogue is illustrated in SEQ ID NO:106 and may be found in GenBank under Accession No. X04666. This sequence encodes an aprotinin analogue having a lysine at position 16 (with reference to the amino acid sequence encoded by SEQ ID NO:106) instead of a valine as found in SEQ ID N0:98. A mutation in the nucleotide sequence of SEQ ID NO:106 may be introduced by methods known in the art to produce the peptide of SEQ ID N0:98 having a valine in position 16.

The polypeptides described herein for inclusion in a protein conjugate may be post-translationally modified, either within a cell in which the polypeptide is expressed or ex vivo by chemical modification. For example, a polypeptide as described herein can be pegylated, acetylated, acylated, amidated, oxidized, cyclized, and/or sulfonated.

Just as the enantiomeric form of the amino acid residues incorporated can alter the stability of a polypeptide, one can modify the length and content of an aprotinin-derived polypeptide to optimize a characteristic such as charge or polarity, hydrophilicity or hydrophobicity, bioavailability, and conjugation properties. For example, one can promote a positive charge by deleting one or more amino acids (e.g., from 1 to about 3 amino acid residues) that are not basic/positively charged or that are less positively charged (e.g., as determined by pKa). Alternatively, or in addition, positive charge can be promoted by adding one or more amino acids (e.g., from 1 to about 3 amino acid residues) that are basic/positively charged or more positively charged (e.g., as determined by pKa) than the residues they replace. One of ordinary skill in the art would recognize that naturally occurring residues have recognized and shared properties, largely attributed to the properties of their side chains. The following information can be used as desired to modify the properties of the aprotinin-derived polypeptides. To increase hydrophobicity, norleucine, methionine, alanine, valine, leucine, isoleucine, histidine, tryptophan, tyrosine, and phenylalanine can be incorporated; to increase neutrality or hydrophilicity, cysteine, serine, and threonine can be incorporated; to increase acidity or negative charge, aspartic acid or glutamic acid can be incorporated; to promote basicity, asparagine, glutamine, histidine, lysine, and arginine can be incorporated. Residues that influence chain orientation include glycine and proline. Residues with aromatic side chains include tryptophan, tyrosine, phenylalanine, and histidine. The polypeptide can therefore vary as described herein with a length ranging having or having at least 6, 7, 8, 9, 10, 11, 12, 13, 14, 15, 16, 17, 18, 19, 20, 21, 25, 35, 50, 75, 100, 200, or 500 amino acids, or any length or range of length between these numbers.

In the reference sequences shown in FIG. 1, the amino acid residues are naturally occurring. Biologically active analogs of these sequences can, however, include one or more non-naturally occurring residues. For example, they may include selenocysteine (e.g., seleno-L-cysteine) at any position, including in the place of cysteine at position 7. Many other “unnatural” amino acid substitutes are known in the art and are available from commercial sources such as the Sigma Aldrich chemical company. Examples of non-naturally occurring amino acids include D-amino acids in most instances, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline. The protein conjugates of the invention encompasses polypeptides and antibody moieties including such residues. It is to be understood that where we refer to a modification or feature such as the inclusion of various enantiomers, a post-translational modification, the inclusion of an “unnatural” residue, or the like, that modification or feature may be present in the part of the conjugate described herein as the aprotinin-derived polypeptide, in the antibody moiety, or in any other amino acid-containing part of the conjugate (e.g., in a detectable label).

Regardless of precise sequence or characteristics of a biologically active fragment or analog of a referenced polypeptide, that variant may have either a comparable, reduced, or enhanced ability to transport an antibody moiety across the BBB relative to the ability of a referenced sequence. For example, where the polypeptide is a biologically active variant of a referenced polypeptide (e.g., of SEQ ID NO:67, 97, or 117), the ability of the variant to transport a conjugated antibody moiety may be reduced, relative to the referenced polypeptide, by at least or about 5% (e.g., by at least or about 5%, 10%, 20%, 25%, 35%, 50%, 60%, 70%, 75%, 80%, 90%, or 95%). Where the polypeptide is a biologically active variant of a referenced polypeptide (e.g., of SEQ ID NO:67, 97, or 117), the ability of the variant to transport a conjugated antibody moiety may be enhanced, relative to the referenced polypeptide, by at least or about 5% (e.g., by at least or about 5%, 10%, 25%, 50%, 100%, 200%, 500%, or 1000%). As noted above, while activity may vary, a biologically active fragment or analog of a referenced polypeptide is one that is active enough to achieve a beneficial result (e.g., a clinically beneficial result in a patient or, on average, a group of patients to whom it is administered). The beneficial result may be a beneficial treatment or diagnostic procedure.

As noted above, the polypeptide can be a fragment or an analog of SEQ ID NO:117 in which at least 13 of the 19 amino acid residues of SEQ ID NO:117 remain invariant. In particular, the analog of SEQ ID NO:117 can include a sequence in which K₁₀ and/or K₁₅ remain invariant. In some embodiments, Asn₁₂ is substituted with another amino acid residue (e.g., Gln); Asn₁₃ is substituted with another amino acid residue (e.g., Gln); and/or Phe₁₄ is substituted with another amino acid residue (e.g., Tyr or Trp). In particular embodiments, the polypeptide can include the sequence of: SEQ ID NO:118; SEQ ID NO:119; SEQ ID NO:120; SEQ ID NO:121; or SEQ ID NO:122.

Where the polypeptide is a fragment of SEQ ID NO:117, or where the polypeptide is an analog of SEQ ID NO:117 that includes a fragment of SEQ ID NO:117, the fragment may have a length of at least 6 amino acid residues (e.g., 6, 7, 8, 9, 10, 11, 12, 13, 14, 15, 16, 17, or 18 amino acid residues) that are identical to 6-18 contiguous amino acid residues of SEQ ID NO:117. In accordance with the present invention, the fragment can be Lys₁₀-Arg₁₁-Asn₁₂-Asn₁₃-Pheₘ-Lys₁₅ (SEQ ID NO:123). Biologically active analogs of SEQ ID NO:17 can have (consist of), or can include (comprise) a sequence in which the lysine residues at positions 10 and 15 are invariant, or a sequence in which the lysine residues at positions 10 and 15 as well as the arginine residue at position 16 is invariant. In the latter case, the biologically active analog would have, or would include, a sequence conforming to the formula Lys-Arg-Xaa₃-Xaa₄-Xaa₅-Lys (SEQ ID NO:106). Xaa₃ can be Asn or Gln; Xaa₄ can be Asn or Gln; and Xaa₅ can be Phe, Tyr, or Trp. The sequence noted above, SEQ ID NO:123, conforms to the generic formula of SEQ ID NO:106. As with other polypeptides described herein, where Xaa₃, Xaa₄, and Xaa₅ are selected, they can be conservative substitutions (i.e., the contiguous Asn residues can be replaced, independently, with Gln, Glu, Asp, or His, and Phe can be replaced with Tyr or Trp). Xaa₃, Xaa₄, and Xaa₅ can also vary in enantiomeric form, can be non-naturally occurring amino acid residues, or can be selected on the basis of another feature or characteristic as described herein.

Antibody Moieties:

In addition to a polypeptide (e.g., an aprotinin-derived polypeptide), the protein conjugates of the present invention include an antibody moiety or a biologically active variant thereof. As noted above, the antibody moiety can be a naturally expressed antibody (e.g., a tetrameric antibody) or a biologically active variant thereof. The antibody moiety can also be a non-naturally occurring antibody (e.g., a single chain antibody or diabody) or a biologically active variant thereof. The variants include, without limitation, a fragment of a naturally occurring antibody (e.g., an Fab fragment or an F(ab′)2 fragment of, e.g., a tetrameric antibody), a fragment of an scFv or diabody, or a variant of a tetrameric antibody, an scFv, a diabody, or fragments thereof that differ by virtue of the addition and/or substitution of one or more amino acid residues. The antibody moiety can be further engineered as, for example, a di-diabody.

As is well known in the art, certain types of antibody fragments can be generated by enzymatic treatment of a “full-length” antibody. Digestion with papain produces two identical Fab fragments, each with a single antigen-binding site, and a residual Fc fragment. The Fab fragment also contains the constant domain of the light chain and the C_{H1} domain of the heavy chain. In contrast, digestion with pepsin yields the F(ab′)₂ fragment that has two antigen-binding sites and is still capable of cross-linking antigen. Antibody moieties incorporated in the present conjugates can be generated by digestion with these enzymes or produced by other methods.

Fab′ fragments, which can also be incorporated, differ from Fab fragments in that they include additional residues at the C-terminus of the C_{H1} domain, including one or more cysteine residues from the antibody hinge region. The cysteine residues of the constant domains bear a free thiol group, which can participate in the conjugation reactions described herein. F(ab′)₂ antibody fragments are pairs of Fab′ fragments linked by cysteine residues in the hinge region. Other chemical couplings of antibody fragments are also known in the art and are useful herein.

The Fv region is a minimal fragment that contains a complete antigen-recognition and binding site consisting of one heavy chain and one light chain variable domain. The three CDRs of each variable domain interact to define an antigen-biding site on the surface of the V_{H}-V_{L} dimer. Collectively, the six CDRs confer antigen-binding specificity to the antibody. As would be known in the art, a “single-chain” antibody or “scFv” fragment is a single chain Fv variant formed when the V_{H} and V_{L} domains of an antibody are included in a single polypeptide chain that recognizes and binds an antigen. Typically, single-chain antibodies include a polypeptide linker between the V_{H} and V_{L} domains that enables the scFv to form a desired three-dimensional structure for antigen binding (see, e.g., Pluckthun, In The Pharmacology of Monoclonal Antibodies, Rosenburg and Moore Eds., Springer-Verlag, New York, 113:269-315, 1994).

In other embodiments, the antibody moiety can be a diabody. Diabodies are small antibody fragments that have two antigen-binding sites. Each fragment contains a V_{H} domain concatenated to a V_{L} domain. However, since the linker between the domains is too short to allow pairing between them on the same chain, the linked V_{H}-V_{L} domains are forced to pair with complementary domains of another chain, creating two antigen-binding sites. Diabodies are described more fully, for example, in EP 404,097; WO 93/11161; and Hollinger et al., Proc. Natl. Acad. Sci. USA 90:6444-6448, 1993.

In other embodiment, the antibody moiety can be a linear antibody. In this case, the antibody moiety is formed from a pair of tandem Fd segments (VH-CH1-VH-CH1) that form a pair of antigen binding regions. Linear antibodies can be bispecific or monospecific as described in, for example, Zapata et al. 1995, Protein Eng. 8(10):1057-1062, 1995.

With respect to targets, the antibody moiety (e.g., a tetrameric antibody, a biologically active variant thereof, an scFv, Fab fragment, Fab′ fragment, or F(ab′)2 fragment, or biologically active variants thereof, regardless of class (i.e., whether of the IgG class or another class) and whether human, humanized, chimeric, polyclonal, monoclonal, or having any other attribute or characteristic described herein) can specifically bind an antigen that is expressed on the cell surface of a dysplasic cell, a tumor cell, or a malignant cell (e.g., a tumor antigen) as well as a growth factor receptor or a cytokine receptor (e.g., an interleukin receptor). The growth factor receptor can be a receptor bound by a member of the epidermal growth factor (EGF) family. Examples of receptors for proteins in the EGF family include an EGF receptor (EGFR), a heparin-binding EGF-like growth factor receptor (HB-EGFR), an amphiregulin receptor (AR), an epiregulin receptor (EPR), an epigen receptor, a betacellulin receptor, and a receptor for a neuregulin (e.g., a receptor for neuregulin-1, neuregulin-2, neuregulin-3, or neuregulin-4). Although the invention is not so limited, when an EGFR is targeted, the antibody moiety can be trastuzumab, cetuximab, or panitumumab, an scFv comprising the variable regions of the heavy and light chains of trastuzumab, cetuximab, or panitumumab, or a biologically active variant of these tetrameric or single chain antibodies. For example, an Fab or F(ab′)2 fragment of trastuzumab, cetuximab, or panitumumab. In other embodiments, the growth factor receptor can be a receptor bound by a member of the vascular endothelial growth factor (VEGF) family. Examples of receptor targets for proteins in the VEGF family include a VEGF receptor (VEGFR; e.g., a receptor for VEGF-A, VEGF-B, VEGF-C, or VEGF-D) or a receptor for placental growth factor (PGFR). As noted, other suitable targets for the antibody moieties in the present protein conjugates are cytokine receptors, including those for members of the tumor necrosis factor (TNF) family. These receptors include those bound by TNF (also known as TNF-α or cachectin), lymphotoxin-α (LT-α), T cell antigen gp39 (CD40L), FASL, 4-1BBL, OX40L, and TNF-related apoptosis inducing ligand (TRAIL). Where the targeted receptor is an interleukin receptor, the antibody moiety can specifically bind an interleukin-2 (IL-2) or interleukin-6 (IL-6) receptor. Although the invention is not so limited, when an antibody moiety targets a receptor for IL-2, the antibody moiety can be basiliximab or daclizumab, an scFV comprising the variable regions of the heavy and light chains of basiliximab or daclizumab, or biologically active variants of tetrameric or single chain antibodies. For example, the antibody moiety can be an Fab or F(ab′)2 fragment of basiliximab or daclizumab. Similarly, when an antibody moiety targets a receptor for IL-6, the antibody moiety can be tocilizumab, an scFV comprising the variable regions of the heavy and light chains of tocilizumab, or a biologically active variant of this tetrameric or single chain antibody (e.g., an Fab or F(ab′)2 fragment of tocilizumab). With respect to function, the antibody moiety can be an anti-cancer agent or an anti-inflammatory agent.

Several of our studies to date, including some of those described in the Examples below, have been conducted with trastuzumab (HERCEPTIN®), which is a monoclonal antibody humanized from the mouse that binds with high affinity to the human epidermal growth factor 2 (HER2)/neu receptor. Trastuzumab is FDA approved for the treatment of breast cancer. The HER2/neu receptor is thought to be an orphan receptor with no known ligand. However, it is expressed on the surface of tumor cells and regulates critical cellular processes such as cell cycle progression, cell survival, cell proliferation, and cell motility. Trastuzumab or any antibody moiety that selectively binds HER2 may be a clinically relevant biomarker for certain tumor types. Therapeutically, when trastuzumab binds to the HER receptor, it not only inhibits the cell's ability to grow and divide, but also marks the cell for destruction by the host's immune system.

Cytotoxins:

A wide variety of cytotoxic agents can be incorporated into the present conjugates. These include cytotoxins that target microtubules, including the taxanes (diterpenes produced by the plants of the genus Taxus) and, in particular, the taxanes currently prescribed as anti-cancer agents (including Taxol® (paclitaxel) and Taxotere® (docetaxel), and Jevtana® (cabazitaxel). Paclitaxel and docetaxel are currently prescribed as antineoplastic agents for treating the patients treatable with the present conjugates (e.g., lung cancer, breast cancer, squamous cell carcinoma of the head and neck, and esophageal, gastric, and colon cancer). Nontaxane microtubule-targeting agents such as an epothilone (e.g., epothilone A, B, C, D, E, or F) and eribulin can also be incorporated.

Other useful cytotoxic agents include the alkaloids (e.g., a vinca alkaloid such as vincristine, vinblastine, vindesine, and vinorelbine), an alkylating agent (e.g., cyclophosphamide, mechlorethamine, chlorambucil, or melphan) an anthracycline (e.g., daunorubicin, doxorubicin, epirubicin, idarubicin, mitoxantrone, and valrubicin), an auristatin (e.g. monomethyl auristatin E (MMAE), an antifolate (e.g., methotrexate or aminopterin), a calicheamicin (e.g., calicheamicin γ 1), a duocarmycin (e.g., adozelesin, bizelesin, or carzelesin); a mitomycin (e.g., mitomycin C), a pyrimidine analog (e.g., fluorouracil), or a derivative of mytansine (e.g., a mytansinoid such as ansamitocin, mertansine, or emtansine).

The cytotoxin can be one that is generally considered less potent, such as doxorubicin, methotrexate, mitomycin, fluorouracil, and the vinca alkaloids (Senter, Curr. Opin. Chem. Biol. 13:235-244, 2009). More potent cytotoxins can also be employed, such as an auristatin (e.g., monomethyl auristatin (MMAE),), a maytansinoid (a derivative of maytansine such as ansamitocin, mertansine, or emtansine), or a calicheamicin (a class of enediyne antibiotic such as calicheamicin γ 1).

### Configurations:

Each part of a given conjugate, including the antibody moiety, cytotoxic agent, linker, and polypeptide, can be selected independently. That is, any of the linkers described herein can be used to conjugate any of the polypeptides and cytotoxic agents described herein to any of the antibody moieties described herein (provided, as one of ordinary skill in the art would understand, that the component parts to be linked include compatible reactive substituents). The conjugates can then be used to deliver the antibody moieties and cytotoxic agents to a patient for treatment of a CNS cancer or other cancer. We may refer to the antibody moiety as a “first agent” and to the cytotoxin as the “second agent.” Where the first agent is an antibody and the second agent is a small molecule drug (e.g., a cytotoxin), the combination of antibody moiety and small molecule drug (e.g., cytotoxin) are a combination therapy for a disease (e.g., a cancer). With the inclusion of a detectable marker, the present conjugates can also be used as imaging agents, providing the means to map the distribution of the targets to which the antibody moieties bind and/or the receptors for which the polypeptides have affinity.

While specific configurations are discussed further below, we note that a given protein conjugate can include one or more polypeptide moieties relative to each antibody moiety (e.g., 1-512 polypeptides relative to each antibody within the conjugate) and one or more cytotoxic agents relative to the polypeptide (e.g., 1-3 cytotoxic agents per polypeptide). As noted, a given protein conjugate is likely to include a single antibody moiety, but it may include two or more (e.g., 2, 3, or 4) that are identical to one another or different from one another. Where different, the antibody moieties may specifically bind the same target or different targets. The component parts of the present conjugates can be configured in a variety of ways. Overall, the conjugate can assume an essentially linear form with an antibody moiety being linked to at least one polypeptide, which is in turn linked to at least one cytotoxic agent. Alternatively, the conjugate can have a branched configuration as seen in dendrimers, with one or more branches extending at some point from the antibody moiety. Where the present conjugates include a branched portion, we may refer to the conjugate as a “dendrimer conjugate” with the understanding that the inclusion of the antibody moiety does not allow for a fully symmetrical dendrimeric form. A dendrimeric conjugate can be structured as in Formula I:

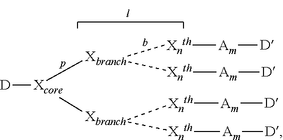

where D is an antibody moiety that is linked to the core moiety of the dendrimer conjugate (X_{core}) either directly (e.g., by way of a bond between a modified residue on the antibody moiety and X_{core}) or indirectly (e.g., by way of a bifunctional linker that joins the antibody moiety to X_{core}). As X_{core} and X_{branch} both join one part of a conjugate to another, we may also refer to either moiety more simply as a “linker”. The complexity of the core moiety can vary, with the number of available extension points, p, varying from 2 to 6, inclusive. Each extension point p can terminate in (and be joined to) a branch moiety, X_{branch}, that, like X_{core}, varies in complexity with each X_{branch} having from 2 to 4 branches, b. Xₙ^{th} is one of n surface branches, and l, an integer from 1 to 5, inclusive, is the number of successive layers of X_{branch} moieties. Where l is 1, each X_{branch} is attached to X_{core}. Where l is more than 1, each X_{branch} distal to the first X_{branch} is attached to another X_{branch}. With regard to the surface branches, Xₙ^{th} is one of n surface branches of the dendrimer. n=p(b)^{l}, and n is typically ≦512 (e.g., ≦500, ≦400, ≦300, ≦200, ≦50, ≦10, or ≦8 branches). To illustrate: where there are two extension points p, where l is 1, and where there are two branches b from each X_{branch}, Xₙ^{th} is 4; where there are three extension points p, where l is 1, and where there are three branches b from each X_{branch}, xₙ^{th} and so forth. Aₘ is a polypeptide as described herein that is attached to a surface branch Xₙ^{th}. In some embodiments, the number of polypeptides Aₘ is less than or equal to the number of surface branches, as each surface branch can be joined to a polypeptide, and some surface branches can be either free of any additional components or joined directly to a cytotoxic agent D′ (i.e., at some surface branches, the polypeptide represented by Aₘ is absent). In some embodiments, the number of polypeptides Aₘ is more than the number of surface branches, as each surface branch can be joined to a first polypeptide that is fused to a second polypeptide of the same or different type in a tail-to-head or head-to-tail configuration. The cytotoxic agent D′ is attached to one or more Aₘ or, as noted, may replace one or more (but not all) Aₘ, attaching directly to one or more Xₙ^{th}. The number of D′ in the dendrimer conjugate can be up to three times the number of polypeptides, as up to three cytotoxic agents can be joined to each polypeptide. The molecular weight of the dendrimer, excluding D, D′ and Aₘ, is ≦500 kilodalton (e.g., ≦500, ≦400, ≦300, ≦200, ≦100, ≦50, or ≦20 kilodaltons).

The linkers employed as X_{core} and X_{branch} can be the same or different, and one can make less complex dendrimer conjugates by employing a bifunctional linker as either X_{core} or X_{branch}. Where X_{core} is a bifunctional linker, p is 1 and the complexity that would have been generated by multiple extensions from X_{core} is missing. This arrangement is illustrated in the Formula below, with the remainder of the conjugate as described above.

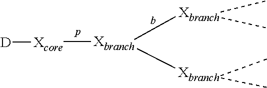

In a variant of this configuration, X_{core} is absent, in which case the antibody moiety is joined directly to an X_{branch}. Where X_{branch}, rather than X_{core}, is a bifunctional linker, b is 1, and the complexity that would have been generated by multiple extensions from X_{branch} is missing. This arrangement is illustrated in the Formula below, with the remainder of the conjugate as described above.

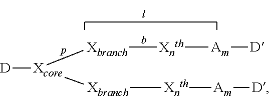

One advantage of the dendrimeric conjugate is the inclusion of multiple surface functionalities to which multiple polypeptides and/or cytotoxins can be conjugated. The ability to alter the complexity of the dendrimeric conjugate allows one to accommodate the various component parts of the protein conjugate. Where X_{core} and X_{branch} are both bifunctional linkers, the conjugate is linear, not dendrimeric.

Methods for synthesizing dendrimers are well known in the art, and the branched portion of the dendrimer (the X_{core} and X_{branch} portions) can also be purchased from a commercial supplier with varying numbers of layers of branches. The dendrimer can be constructed by known methods of either divergent synthesis or convergent synthesis. In the former, the dendrimer is assembled from its core, extending outwardly by a series of reactions. In the latter, the dendrimer is assembled from small molecules, which end up at the periphery of the structure as the reaction proceeds inwardly. The advantages of each approach are appreciated in the art. Alternatively, the dendrimers can be synthesized by click chemistry, employing Diels-Alder reactions, thiol-yne reactions, and azide-alkyne reactions.

Any given dendrimer can be synthesized to have different functionalities in the core and in the branches to control properties such as solubility, thermal stability, and attachment of compounds for particular applications. Synthetic processes can also precisely control the size, number of branches, numbers of layers of branches from the core, and the functionalities of the terminal branches for attachment of various reactive groups.

The dendrimer part of the conjugate may include, as a core moiety: propargylamine, ethylenediamine, triethanolamine, pentaerythritol, tetraphenyl methane, azido-propyl(alkyl)amine, hydroxyethyl(alkyl)amine, trimesoylchloride, diamino hexane, diaminobutane, cystamine, propylenediamine, and derivatives of any of the foregoing. These core moieties can be used to synthesize the poly(amido amine) (PAMAM) dendrimer. Lysine can also be used as a core moiety to synthesize a polylysine dendrimer. Alternatively, the compound can include a propyleneimine to synthesize a POPAM dendrimer.

The conjugates of the invention can have, as branch moieties: propargylamine, ethylene-diamine, triethanolamine, pentaerythritol, propylamine, propyleneimine, azido-propyl(alkyl)amine, hydroxyethyl(alkyl)amine, tetraphenyl methane, trimesoylchloride, diamino hexane, diamino-butane, cystamine, propylenediamine, and lysine or a derivative of any one of the foregoing.

The surface branches of dendrimers can be functionalized for conjugation with polypeptides derivatized with appropriate reactive groups. For example, the surface branches can be functionalized with linkers such as N-succinimidyl 3-2-pyridyldithio (SPDP) to generate a dendrimer-pyridyl-disulfide intermediate that can then react with a polypeptide containing a cysteine residue (or other —SH bearing group). Alternatively, the surface branches of dendrimers can be functionalized with the linkers N-succinimidyl S-acetylthioacetate (SATA) or N-succinimidyl-S-acetylthiopropionate (SATP) to form a dendrimer-sulfydryl intermediate that can be reacted with a maleimide derivatized polypeptide. SATA and SATP are reactive toward amines and add protected sulihydryls groups, resulting in an amine to sulfur conjugation (as described further below).

Linkers:

A given linker within the present compositions can provide a cleavable linkage (e.g., a thioester linkage) or a non-cleavable linkage (e.g., a maleimide linkage). For example, a cytotoxic protein can be bound to a linker that reacts with modified free amines, which are present at lysine residues within the polypeptide and/or at the amino-terminus of the polypeptide. Thus, linkers useful in the present conjugates can comprise a group that is reactive with a primary amine on the polypeptide or modified polypeptide to which the antibody moiety is conjugated. More specifically, the linker can be selected from the group consisting of monofluoro cyclooctyne (MFCO), bicyclo[6.1.0]nonyne (BCN), N-succinimidyl-S-acetylthioacetate (SATA), N-succinimidyl-S-acetylthiopropionate (SATP), maleimido and dibenzocyclooctyne ester (a DBCO ester). Useful cyclooctynes, within a given linker, include OCT, ALO, MOFO, DIFO, DIBO, BARAC, DIBAC, and DIMAC.

The components of the conjugates can be conjugated through a variety of linking groups (linkers), e.g., sulfhydryl groups, amino groups (amines), or any appropriate reactive group. The linker can be a covalent bond. Homobifunctional and hetero-bifunctional cross-linkers (conjugation agents) useful in the present conjugates are available from many commercial sources. Although known in the art, we note briefly that the reactive groups in homobifunctional crosslinkers are identical and positioned at opposite ends of the linker (e.g., a crosslinker's spacer arm). They are convenient to work with, as the reaction can be completed with a one-step chemical crosslink, and they can be assembled where desired to form dimers and polymers. Heterobifunctional crosslinkers have two distinct groups, which allows the conjugation to progress as a two-step reaction. These linkers are also commercially available in different lengths with different types of spacer arms. Conjugation can proceed between primary amine groups (e.g., on a lysine residue) and sulfhydryl groups (e.g., on a cysteine residue). Linkers having a greater number of reactive groups, whether the same or different, can be used to link more than two entities. For example, a homo- or heterotrifunctional linker can link one antibody moiety with two polypeptides.

Among the commercially available homobifunctional cross-linkers are: BSOCOES (Bis(2-[Succinimidooxycarbonyloxy]ethyl) sulfone; DPDPB (1,4-Di-(3′-[2pyridyldithio]-propionamido) butane; DSS (disuccinimidyl suberate); DST (disuccinimidyl tartrate); Sulfo DST (sulfodisuccinimidyl tartrate); DSP (dithiobis(succinimidyl propionate); DTSSP (3,3′-Dithiobis(sulfosuccinimidyl propionate); EGS (ethylene glycol bis(succinimidyl succinate)); and BASED (Bis(β-[4-azidosalicylamido]-ethyl)disulfide iodinatable).

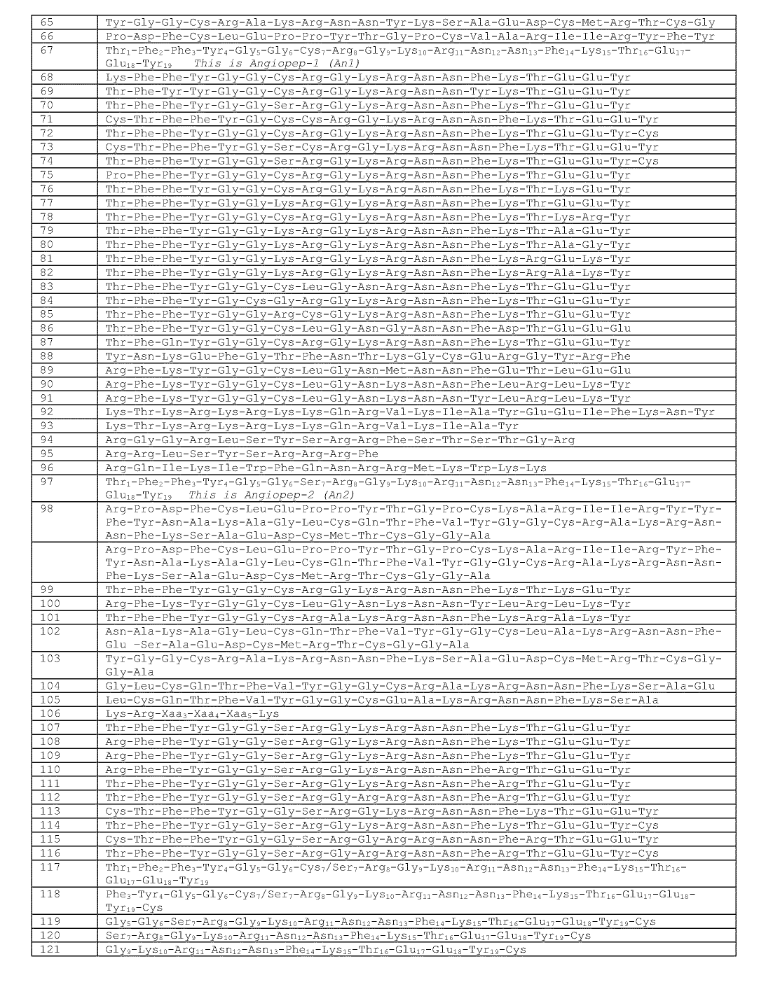

Sites available for cross-linking may be found on the polypeptides. The linker group may be or may comprise a flexible arm having, e.g., 2, 3, 4, 5, 6, 7, 8, 9, 10, 11, 12, 13, 14, 15, or 20 carbon atoms in, e.g., an aliphatic chain. An aliphatic linker is illustrated in FIG. 2 (e.g., with an amide bond to the polypeptide and an ester bond to the cytotoxic agent). As noted, where an aliphatic linker is used, it may vary with regard to length (e.g. C₁-C₂₀) and the chemical moieties it includes (e.g., an amino group or carbamate). Generally, linkers considered to have a short arm have a <2-carbon carbon chain. Medium-sized arms have a carbon chain of 2-5 carbon atoms, and long-armed linkers have six or more carbons in a chain. Exemplary linkers include pyridinedisulfide, thiosulfonate, vinylsulfonate, isocyanate, imidoester, diazine, hydrazine, thiol, carboxylic acid, multi-peptide linkers, and acetylene. Alternatively, other linkers than can be used include BS³ [Bis(sulfosuccinimidyl)-suberate] (which is a homobifunctional N-hydroxy-succinimide ester that targets accessible primary amines), NHS/EDC (N-hydroxysuccinimide and 1-ethyl-3-(3-dimethylaminopropyl)-carbodiimide (NHS/EDC allows for the conjugation of primary amine groups with carboxyl groups), sulfo-EMCS ([N-ε-maleimidocaproic acid]hydrazide (sulfo-EMCS are heterobifunctional reactive groups that are reactive toward sulfhydryl and amino groups), hydrazide (most proteins contain exposed carbohydrates and hydrazide is a useful reagent for linking carboxyl groups to primary amines). Regions available for cross-linking with a homo- or heterobifunctional cross linker may be found on the polypeptides of the present invention. Another useful linker is SATA (N-succinimidyl-S-acetylthioacetate), which is reactive towards amines and adds protected sulfhydryl groups. With respect to the method used, the process of conjugating an antibody moiety to the polypeptide preferably does not alter or change the key characteristics of the antibody moiety, such as its immunospecificity or immunoreactivity. Homobifunctional amine-specific cross linkers can rely on NHS-ester and imidoester reactive groups for selective conjugation of primary amines and may be cleavable.

To form covalent bonds, one can use as a chemically reactive group a wide variety of active carboxyl groups (e.g., esters) where the hydroxyl moiety is physiologically acceptable at the levels required to modify the peptide. Particular agents include N-hydroxysuccinimide (NHS), N-hydroxy-sulfosuccinimide (sulfo-NHS), maleimide-benzoyl-succinimide (MBS), gamma-maleimido-butyryloxy succinimide ester (GMBS), maleimido propionic acid (MPA), maleimido hexanoic acid (MHA), and maleimido undecanoic acid (MUA).

Primary amines are the principal targets for NHS esters. Accessible α-amine groups present on the N-termini of proteins and the ε-amine of lysine react with NHS esters. Thus, compounds of the invention can include a linker having a NHS ester conjugated to an N-terminal amino of a peptide or to an ε-amine of lysine. An amide bond is formed when the NHS ester reacts with primary amines releasing N-hydroxysuccinimide. We may refer to these succinimide containing reactive groups more simply as succinimidyl groups. In some embodiments, the functional group on the protein will be a thiol group and the chemically reactive group will be a maleimido-containing group such as gamma-maleimide-butylamide (GMBA or MPA). Such maleimide-containing groups may be referred to herein as maleido groups.

The maleimido group is most selective for sulfhydryl groups on peptides when the pH of the reaction mixture is 6.5-7.4. At pH 7.0, the rate of reaction of maleimido groups with sulfhydryls (e.g., thiol groups on proteins such as serum albumin or IgG) is 1000-fold faster than with amines. Thus, a stable thioether linkage between the maleimido group and the sulfhydryl can be formed. Accordingly, a compound of the invention can include a linker having a maleimido group conjugated to a sulfhydryl group of a polypeptide. Amine-to-amine linkers include NHS esters, imidoesters, and others, examples of which are listed in the Table below.

Exemplary NHS esters:
DSG (disuccinimidyl glutarate)
DSS (disuccinimidyl suberate)
BS³ (bis[sulfosuccinimidyl] suberate)
TSAT (tris-succinimidyl aminotriacetate)
Variants of bis-succinimide ester-activated compounds including a
polyethylene glycol spacer such as BS(PEG)ₙ where n is 1-20
(e.g., BS(PEG)₅ and BS(PEG)₉)
DSP (Dithiobis[succinimidyl propionate])
DTSSP (3,3′-dithiobis[sulfosuccinimidylpropionate])
DST (disuccinimidyl tartarate)
BSOCOES (bis[2-(succinimidooxycarbonyloxy)ethyl]sulfone)
EGS (ethylene glycol bis[succinimidylsuccinate])
sulfo-EGS (ethylene glycol bis[sulfosuccinimidylsuccinate])
Exemplary imidoesters:
DMA (dimethyl adipimidate•2HCl)
DMP (dimethyl pimelimidate•2HCl)
DMS (dimethyl suberimidate•2HCl)
DTBP (dimethyl 3,3′-dithiobispropionimidate•2HCl)
Other exemplary amine-to-amine linkers:
DFDNB (1,5-difluoro-2,4-dinitrobenzene)
THPP (β-[tris(hydroxymethyl) phosphino] propionic acid (betaine))

The linker may also be a sulfhydryl-to-sulfhydryl linker, such as the maleimides and pyridyldithiols listed in the Table below.

Exemplary maleimides:
BMOE (bis-maleimidoethane)
BMB (1,4-bismaleimidobutane)
BMH (bismaleimidohexane)
TMEA (tris[2-maleimidoethyl]amine)
BM(PEG)2 1,8-bis-maleimidodiethyleneglycol)
BM(PEG)ₙ, where n is 1 to 20 (e.g., 2 or 3)
BMDB (1,4 bismaleimidyl-2,3-dihydroxybutane)
DTME (dithio-bismaleimidoethane)
Exemplary pyridyldithiol:
DPDPB (1,4-di-[3′-(2′-pyridyldithio)-propionamido]butane)
Another sulfhydryl linker:
HBVS (1,6-hexane-bis-vinylsulfone)

The linker may be an amine-to-sulfhydryl linker, which includes NHS ester/maliemide compounds. Examples of these compounds are provided in the Table below.

Amine-to-sulfhydryl linkers:

AMAS (N-(α-maleimidoacetoxy)succinimide ester)
BMPS (N-[β-maleimidopropyloxy]succinimide ester)
GMBS (N-[γ-maleimidobutyryloxy]succinimide ester)
sulfo-GMBS (N-[γ-maleimidobutyryloxy]sulfosuccinimide ester)
MBS (m-maleimidobenzoyl-N-hydroxysuccinimide ester)
sulfo-MBS (m-maleimidobenzoyl-N-hydroxysulfosuccinimide ester)
SMCC (succinimidyl 4-[N-maleimidomethyl]cyclohexane-1-carboxylate)
sulfo-SMCC (Sulfosuccinimidyl 4-[N-maleimidomethyl]cyclohexane-1-
carboxylate)
EMCS ([N-ε-maleimidocaproyloxy]succinimide ester)
Sulfo-EMCS ([N-ε-maleimidocaproyloxy]sulfosuccinimide ester)
SMPB (succinimidyl 4-[p-maleimidophenyl]butyrate)
sulfo-SMPB (sulfosuccinimidyl 4-[p-maleimidophenyl]butyrate)
SMPH (succinimidyl-6-[β-maleimidopropionamido]hexanoate)
LC-SMCC (succinimidyl-4-[N-maleimidomethyl]cyclohexane-1-carboxy-
[6-amidocaproate])
sulfo-KMUS (N-[κ-maleimidoundecanoyloxy]sulfosuccinimide ester)
SM(PEG)ₙ (succinimidyl-([N-maleimidopropionamido-polyethyleneglycol)
ester), where n is 1 to 30 (e.g., 2, 4, 6, 8, 12, or 24)
SPDP (N-succinimidyl 3-(2-pyridyldithio)-propionate)
LC-SPDP (succinimidyl 6-(3-[2-pyridyldithio]-propionamido)hexanoate)
sulfo-LC-SPDP (sulfosuccinimidyl 6-(3′-[2-pyridyldithio]-
propionamido)hexanoate)
SMPT (4-succinimidyloxycarbonyl-α-methyl-α-[2-pyridyldithio]toluene)
Sulfo-LC-SMPT (4-sulfosuccinimidyl-6-[α-methyl-α-
(2-pyridyldithio)toluamido]hexanoate)
SIA (N-succinimidyl iodoacetate)
SBAP (succinimidyl 3-[bromoacetamido]propionate)
SLAB (N-succinimidyl[4-iodoacetyl]aminobenzoate)
sulfo-SIAB (N-sulfosuccinimidyl[4-iodoacetyl]aminobenzoate)

The linker can react with an amino group and a non-selective entity. Such linkers include NHS ester/aryl azide and NHS ester/diazirine linkers, examples of which are listed in the Table below.

NHS ester/aryl azide linkers:
NHS-ASA (N-hydroxysuccinimidyl-4-azidosalicylic acid)
ANB-NOS (N-5-azido-2-nitrobenzoyloxysuccinimide)
sulfo-HSAB (N-hydroxysulfosuccinimidyl-4-azidobenzoate)
sulfo-NHS-LC-ASA (sulfosuccinimidyl[4-azidosalicylamido]hexanoate)
SANPAH (N-succinimidyl-6-(4′-azido-2′-nitrophenylamino)hexanoate)
sulfo-SANPAH (N-sulfosuccinimidyl-6-(4′-azido-2′-
nitrophenylamino)hexanoate)
sulfo-SFAD (sulfosuccinimidyl-(perfluoroazidobenzamido)-ethyl-1,3′-
dithioproprionate)
sulfo-SAND (sulfosuccinimidyl-2-(m-azido-o-nitrobenzamido)-ethyl-1,3′-
proprionate)
sulfo-SAED (sulfosuccinimidyl 2-[7-amino-4-methylcoumarin-3-
acetamido]ethyl-1,3′dithiopropionate)
NHS ester/diazirine linkers:
SDA (succinimidyl 4,4′-azipentanoate)
LC-SDA (succinimidyl 6-(4,4′-azipentanamido)hexanoate)
SDAD (succinimidyl 2-([4,4′-azipentanamido]ethyl)-1,3′-
dithioproprionate)
sulfo-SDA (sulfosuccinimidyl 4,4′-azipentanoate)
sulfo-LC-SDA (sulfosuccinimidyl 6-(4,4′-azipentanamido)hexanoate)
sulfo-SDAD (sulfosuccinimidyl 2-([4,4′-azipentanamido]ethyl)-1,3′-
dithioproprionate)

Exemplary amine-to-carboxyl linkers include carbodiimide compounds (e.g., DCC (N,N-dicyclohexylcarbodimide) and EDC (1-ethyl-3-[3-dimethylaminopropyl]carbodiimide)). Exemplary sulfhydryl-to-nonselective linkers include pyridyldithiol/aryl azide compounds (e.g., APDP ((N-[4-(p-azidosalicylamido)butyl]-3′-(2′-pyridyldithio)propionamide)). Exemplary sulfhydryl-to-carbohydrate linkers include maleimide/hydrazide compounds (e.g., BMPH (N-[β-maleimidopropionic acid]hydrazide), EMCH ([N-ε-maleimidocaproic acid]hydrazide), MPBH 4-(4-N-maleimidophenyl)butyric acid hydrazide), and KMUH (N-[κ-maleimidoundecanoic acid]hydrazide)) and pyridyldithiol/hydrazide compounds (e.g., PDPH (3-(2-pyridyldithio)propionyl hydrazide)). Exemplary carbohydrate-to-nonselective linkers include hydrazide/aryl azide compounds (e.g., ABH (p-azidobenzoyl hydrazide)). Exemplary hydroxyl-to-sulfhydryl linkers include isocyanate/maleimide compounds (e.g., (N-[p-maleimidophenyl]isocyanate)). Exemplary amine-to-DNA linkers include NHS ester/psoralen compounds (e.g., SPB (succinimidyl-[4-(psoralen-8-yloxy)]-butyrate)).

To generate a branch point of varying complexity in a protein conjugate, the linker can be capable of linking 3-7 entities.

Exemplary tri-functional linkers:

| | | |
|---|---|---|
| TMEA; Tris-(2-maleimidoethyl)amine | 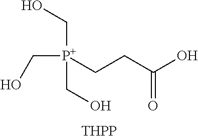 | LC-TSAT (tris-succinimidyl (6- aminocaproyl)aminotriacetate, tris-succinimidyl-1,3,5- benzenetricarboxylate |
| 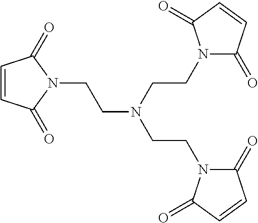 [SMILES: O=C1C=CC(=O)N1CCN(CCN1C(=O)C=CC1=O)CCN1C(=O)C=CC1=O] | | MDSI (maleimido-3,5- disuccinimidyl isophthalate) |

| | |
|---|---|
| TSAT; Tris-succinimidyl aminotriacetate 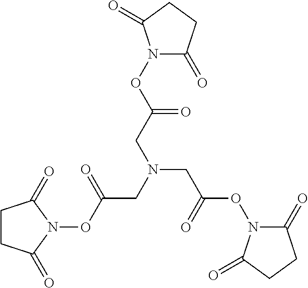 [SMILES: O=C(CN(CC(=O)ON1C(=O)CCC1=O)CC(=O)ON1C(=O)CCC1=O)ON1C(=O)CCC1=O] | SDMB (succinimidyl-3,5-dimaleimido- phenyl benzoate Mal-4 (tetrakis-(3-maleimidopropyl)pentaerythritol, NHS-4 (tetrakis-(N-succinimidyl- carboxypropyl)pentaerythritol)) |

TMEA and TSAT reach through their maleimide groups with sulfhydryl groups. The hydroxyl groups and carboxy group of THPP can react with primary or secondary amines. Other useful linkers conform to the formula Y═C═N-Q-A-C(O)—Z, where Q is a homoaromatic or heteroaromatic ring system; A is a single bond or an unsubstituted or substituted divalent C₁₋₃₀ bridging group, Y is O or S; and Z is Cl, Br, I, N₃, N-succinimidyloxy, imidazolyl, 1-benzo-triazolyloxy, OAr where Ar is an electron-deficient activating aryl group, or OC(O)R where R is -A-Q-N═C═Y or C₄-20 tertiary-alkyl (see U.S. Pat. No. 4,680,338).

Other useful linkers have the formula

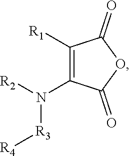

where R₁ is H, C₁₋₆ alkyl, C₂₋₆ alkenyl, C₆₋₁₂ aryl or aralkyl or these coupled with a divalent organic —O—, —S—, or

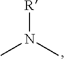

where R′ is C₁₋₆ alkyl, linking moiety; R₂ is H, C₁₋₁₂ alkyl, C₆₋₁₂ aryl, or C₆₋₁₂ aralkyl, R₃ is

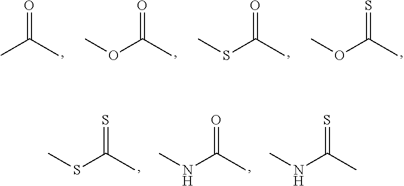

or another chemical structure that is able to delocalize the lone pair electrons of the adjacent nitrogen and R₄ is a pendant reactive group capable of linking R₃ to a peptide vector or to an agent (see U.S. Pat. No. 5,306,809).

A given linker useful in the present conjugates may also include at least one amino acid residue and can be a peptide of at least or about 2, 3, 4, 5, 6, 7, 10, 15, 20, 25, 30, 40, or 50 amino acid residues. Where the linker is a single amino acid residue it can be any naturally or non-naturally occurring amino acid (e.g., Gly, Cys, Lys, Glu, or Asp) or a di-peptide including two such residues (e.g., Gly-Lys). Where the linker is a short peptide, it can be a glycine-rich peptide (which tend to be flexible) such as a peptide having the sequence [Gly-Gly-Gly-Gly-Ser]ₙ (SEQ ID NO:129) where n is an integer from 1 to 6, inclusive (see U.S. Pat. No. 7,271,149) or a serine-rich peptide linker (see U.S. Pat. No. 5,525,491). Serine rich peptide linkers include those of the formula [X-X-X-X-Gly]_{y} (SEQ ID NO:130) where up to two of the X are Thr, the remaining X are Ser, and y is an integer from 1 to 5, inclusive (e.g., Ser-Ser-Ser-Ser-Gly (SEQ ID NO:131), where y is greater than 1). Other linkers include rigid linkers (e.g., PAPAP (SEQ ID NO:132) and (PT)ₙP (SEQ ID NO:133), where n is 2, 3, 4, 5, 6, or 7) and α-helical linkers (e.g., A(EAAAK)ₙA (SEQ ID NO:134), where n is 1, 2, 3, 4, or 5). When the linker is succinic acid, one carboxyl group thereof may form an amide bond with an amino group of the amino acid residue, and the other carboxyl group thereof may, for example, form an amide bond with an amino group of the peptide or substituent. When the linker is Lys, Glu, or Asp, the carboxyl group thereof may form an amide bond with an amino group of the amino acid residue, and the amino group thereof may, for example, form an amide bond with a carboxyl group of the substituent. When Lys is used as the linker, a further linker may be inserted between the ε-amino group of Lys and the substituent. The further linker may be succinic acid, which can form an amide bond with the ε-amino group of Lys and with an amino group present in the substituent. In one embodiment, the further linker is Glu or Asp (e.g., which forms an amide bond with the ε-amino group of Lys and another amide bond with a carboxyl group present in the substituent), that is, the substituent is a N^{ε}-acylated lysine residue.

The peptide linker can also be a branched polypeptide. Exemplary branched peptide linkers are described in U.S. Pat. No. 6,759,509. Such linkers include those of the formula:

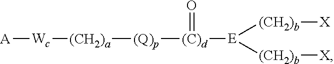

where A is a thiol acceptor; W is a bridging moiety; c is an integer of 0 to 1; a is an integer of 2 to 12; Q is O, NH, or N-lower alkyl; p is an integer of 0 or 1; d is an integer of 0 or 1; E is a polyvalent atom; each b is an integer of 1 to 10; each X is of the formula:

—CO—Y—Zₘ—Gₙ,

where Y is two amino acid residues in the L form; Z is one or two amino acid residues; m is an integer of 0 or 1; G is a self-immolative spacer; and n is a integer of 0 or 1; provided that when n is 0 then —Y—Zₘ is Ala-Leu-Ala-Leu or Gly-Phe-Leu-Gly; or each X is of the formula:

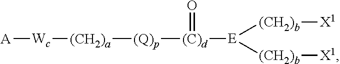

where each X¹ is of the formula —CO—Y—Zₘ—Gₙ; and where Y, Z, Q, E, G, m, d, p, a, b, and n are as defined above; or each X¹ is of the formula:

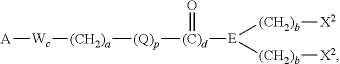

where each X² is of the formula —CO—Y—Zₘ—Gₙ; and where Y, Z, G, Q, E, m, d, p, a, b, and n are as defined above; or each X² is of the formula:

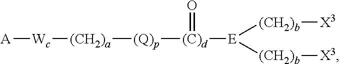

where each X³ is of the formula —CO—Y—Zₘ—Gₙ; and wherein Y, Z, G, Q, E, m, d, p, a, b, and n are as defined above; or each X³ is of the formula

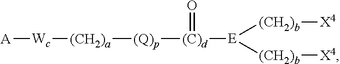

where each X⁴ is of the formula —CO—Y—Zₘ—Gₙ; and where Y, Z, G, Q, E, m, d, p, a, b, and n are as defined above.

The branched linker may employ an intermediate self-immolative spacer moiety (G), which covalently links together the agent or peptide vector and the branched peptide linker. A self-immolative spacer can be a bifunctional chemical moiety capable of covalently linking together two chemical moieties and releasing one of said spaced chemical moieties from the tripartate molecule by means of enzymatic cleavage (e.g., any appropriate linker described herein. In certain embodiments, G is a self-immolative spacer moiety which spaces and covalently links together the agent or peptide vector and the peptide linker, where the spacer is linked to the peptide vector or agent via the T moiety (as used in the following formulas “T” represents a nucleophilic atom which is already contained in the agent or peptide vector), and which may be represented by

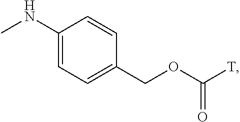

where T is O, N or S; —HN—R¹—COT, where T is O, N or S, and R¹ is C₁₋₅ alkyl;

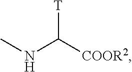

where T is O, N, or S, and R² is H or C₁₋₅ alkyl;

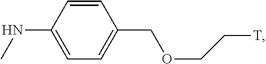

where T is O, N or S; or

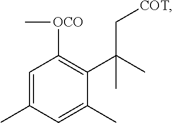

where T is O, N, or S. Preferred Gs include PABC (p-aminobenzyl-carbamoyl), GABA (γ-aminobutyric acid), α,α-dimethyl GABA, and β,β-dimethyl GABA.

In the branched linker, the thiol acceptor “A” is linked to a peptide vector or agent by a sulfur atom derived from the peptide vector or agent. The thiol acceptor can be, for example, an α-substituted acetyl group. Such a group has the formula:

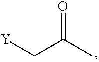

where Y is a leaving group such as Cl, Br, I, mesylate, tosylate, and the like. If the thiol acceptor is an alpha-substituted acetyl group, the thiol adduct after linkage to the ligand forms the bond —S—CH₂ Preferably, the thiol acceptor is a Michael Addition acceptor. A representative Michael Addition acceptor of this invention has the formula

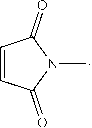

After linkage the thiol group of the ligand, the Michael Addition acceptor becomes a Michael Addition adduct, e.g.,

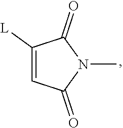

where L is an agent or peptide vector.

The bridging group “W” is a bifunctional chemical moiety capable of covalently linking together two spaced chemical moieties into a stable tripartate molecule. Examples of bridging groups are described in S. S. Wong, Chemistry of Protein Conjugation and Crosslinking. CRC Press, Florida, (1991); and G. E. Means and R. E. Feeney, Bioconiugate Chemistry, vol. 1, pp. 2-12, (1990), the disclosures of which are incorporated herein by reference. W can covalently link the thiol acceptor to a keto moiety. An exemplary bridging group has the formula —(CH₂)_{f} (Z)_{g}—(CH₂)ₕ—, where f is 0 to 10; h is 0 to 10; g is 0 or 1, provided that when g is 0, then f+h is 1 to 10; Z is S, O, NH, SO₂, phenyl, naphthyl, a polyethylene glycol, a cycloaliphatic hydrocarbon ring containing 3 to 10 carbon atoms, or a heteroaromatic hydrocarbon ring containing 3 to 6 carbon atoms and 1 or 2 heteroatoms selected from O, N, or S. Preferred cycloaliphatic moieties include cyclopropyl, cyclobutyl, cyclopentyl, cyclohexyl, and the like. Preferred heteroaromatic moieties include pyridyl, polyethlene glycol (1-20 repeating units), furanyl, pyranyl, pyrimidinyl, pyrazinyl, pyridazinyl, oxazinyl, pyrrolyl, thiazolyl, morpholinyl, and the like. In the bridging group, it is preferred that when g is 0, f+h is an integer of 2 to 6 (e.g., 2 to 4 such as 2). When g is 1, it is preferred that f is 0, 1 or 2; and that h is 0, 1 or 2. Preferred bridging groups coupled to thiol acceptors are shown in the Pierce Catalog, pp. E-12, E-13, E-14, E-15, E-16, and E-17 (1992).

Modified Polypeptides:

Any of the polypeptides incorporated in the protein conjugates of the invention can be modified to chemically interact with, or to include, a linker as described herein. These modified polypeptides and peptide-linker constructs are within the scope of the present invention and may be packaged as a component of a kit with instructions for completing the process of conjugation to an antibody (e.g., a linker-bound antibody moiety) or a cytotoxin. For example, a polypeptide can be modified to include an N₃ azide group, which will react with an alkyne in a linker bound to an antibody moiety (and vice versa; the polypeptide can be bound to a linker including an alkyne, which will react with an azide group extending from an antibody moiety). In other embodiments, and by way of illustration only, the polypeptide can be modified to include a cysteine residue or other thiol-bearing moiety (e.g., C—SH) at the N-terminus, the C-terminus, or both, for reaction with, for example, a maleimide-containing linker such as Mal-AMCHC-OSu or SPDP. As noted, the polypeptides incorporated in the protein conjugates can be conjugated to cytotoxic agents, and polypeptides conjugated to cytotoxic agents are within the scope of the present invention.

Antibody-Polypeptide-Cytotoxin Conjugates and Methods of Making Same:

Preferred conjugation techniques include the cross linker SATA and application of click chemistry. SATA is a heterobifunctional cross linker that facilitates the formation of a covalent bond to link two molecules (e.g., an antibody moiety to the polypeptide moiety). The succinimidyl ester reacts with a primary amine to introduce a thiol group into the molecule followed by the removal of the acetyl group, thereby generating a sulihydryl. The thiol group provides the target to link the two moieties together via a disulfide bond. One of the advantages of employing SATA is that the modified molecule can be stored for long periods of time for later conjugation reactions because the sulihydryl groups can be added in a protected form (which forms are within the scope of the present invention).

As noted, copper-free click chemistry techniques may be used to produce the conjugates described herein. Click chemistry is generally understood as a modular reaction that is widely applicable, stereospecific, and capable of producing high yields of products under mild conditions (e.g., physiological conditions). Click chemistry has been described as encompassing four classes of chemical transformations. The first are non-aldol type carbonyl chemical reactions, such as those that form ureas, thioureas, oxime ethers, hydrazone, amides, and aromatic heterocycles. The second transformations are nucleophilic substitution reactions in which a ring within a strained heterocyclic electrophile (e.g., epoxides, aziridines and aziridinium ions) is opened. In the third, addition reactions to C-C multiples bonds, such as Michael addition, epoxidation, aziridation, and dihydroxylation occurs, and in the fourth, are cycloaddition reactions, such as 1,3-dipolar cycloaddition and Diels-Alder reactions. 1,3-dipolar cycloaddition (1,3-Huisgen reaction) of an alkyne and an azide to form five membered triazole is a particular example of a click reaction. See, Guddehalli Parameswarappy, Sharavathi, “Bifunctional cyclooctynes in copper-free click chemistry for applications in radionuclide chemistry nd 4-Alkylpyridine derivatives in intramolecular dearomatization and heterocycle synethsis”, Dissertation, University of Iowa, 2011. http://ir.uiowa.edu/etd/2710. Click chemistry methods may utilize the highly toxic catalyst copper in production methods that produce high yields. Preliminary conjugation reactions showed, however, that the overall incorporation of a polypeptide in a conjugate of the invention was very low (6%). The conjugate was also unstable, and a precipitation of the product was observed. Therefore, the conjugation steps using click chemistry were optimized to eliminate the use of copper. Briefly, step one involves the activating groups for conjugation as well as purification and dialysis of the intermediate. For this step, a first component (e.g., the antibody or antibody fragment) reacts with a linker generating a first component-linker intermediate (e.g., an antibody- or antibody fragment-linker intermediate). Next, the linker having an activated group for conjugation (i.e., alkyne) and a second component (e.g., a polypeptide having an azide) react forming the desired conjugate which can be further purified. This second step is efficient because the reaction between the relevant groups (alkyne and azide moieties) takes place within 24 hours at room temperature and without denaturing the protein. Neither step in this procedure interferes with the biological activity of the molecules generated.

Alternatively, the polypeptide moiety may be attached or conjugated to a carbohydrate moiety of the antibody molecule through an oxidation process. The method of carbohydrate oxidation can be chemical or enzymatic. The carbohydrate moiety can be located on the Fc region of the antibody, Fab, or Fab′ fragments. Oxidation of the Fc region of the antibody moiety can be carried out using known methods to produce an aldehyde. Oxidizing agents can be selected from the group consisting of periodic acid, paraperiodic acid, sodium metaperiodate and potassium metaperiodate. This step is followed by a reaction with an amine group selected from the group consisting of ammonia derivatives such as primary amine, secondary amine, hydroxylamine, hydrazine, hydrazide, phenylhydrazine, semicarbazide or thiosemicarbazide). The enzymatic method involves reacting the carbohydrate moiety of the antibody molecule with an enzyme such as galactose oxidate in the presence of oxygen to form an aldehyde.

Manufacturing compounds of the invention: The invention features methods to synthesize the protein conjugates described herein. The dendrimer can be conjugated to multiple polypeptides via reactive groups on surface branches. For example, one can react a dendrimer with N-Succinimidyl 3-(2-pyridyldithio)-propionate to form a dendrimer-pyridyl-disulfide intermediate and then react that intermediate with polypeptides containing cysteine residues to attach a polypeptide to each of the surface branches. Alternatively, one can react the dendrimer with N-succinimidyl S-acetylthioacetate to form a dendrimer-sulfydryl intermediate followed by a reaction with a maleimide derivative of the polypeptide to form a dendrimer-polypeptide complex.

The dendrimer-polypeptide complex is then reacted with a first agent as described above and the resulting dendrimer-polypeptide-first agent complex can be produced in a pharmaceutically acceptable form (e.g., as a pharmaceutically acceptable salt).

Alternatively, one can first react the dendrimer with an antibody moiety via a functional group (e.g., azide), and the surface branches of the resulting dendrimer-antibody moiety complex can be functionalized to attach a cytotoxin or a polypeptide-linked cytotoxin to the termini of the surface branches.

The methods of manufacturing conjugates of the invention may additionally involve attachment of any of the linkers described above to the dendrimer prior to attachment of the polypeptides or the antibody moiety.

Assessment: The protein conjugates of the present invention can be assessed in any number of ways. For example, a protein conjugate can be assessed for BBB permeability (by in situ brain perfusion or testing in an ex vivo model of the BBB such as the model described in U.S. Pat. No. 7,557,182); for the affinity of the antibody moiety for its target; for cytotoxicity (e.g., by BT-474 [³H]-thymidine incorporation); for solubility and/or stability in vitro; for purity (e.g., low levels of unconjugated antibody moieties and low levels of protein aggregates can be confirmed by gel separation and Western blotting); and for stability and tissue distribution in vivo (e.g., by measuring plasma levels over time and tissue distribution by imaging assays).

The present methods related to synthesis of a conjugate as described herein can be readily modified to produce a pharmaceutically acceptable salt of the conjugate. Pharmaceutical compositions including such salts and methods of administering them are accordingly within the scope of the present invention.

Pharmaceutical compositions: The present invention also features pharmaceutical compositions that contain a therapeutically effective amount of a protein conjugate of the invention. The compositions can be formulated for administration by any of a variety of routes of administration, and can include one or more physiologically acceptable excipients, which may vary depending on the route of administration. We use the term “excipient” broadly to mean any compound or substance, including those that may also be referred to as “carriers” or “diluents.” Preparing pharmaceutical and physiologically acceptable compositions is generally considered to be routine in the art, and one of ordinary skill in the art can consult numerous authorities for guidance. For example, one can consult Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions of the present invention can be prepared for oral or parenteral administration, although we expect parenteral administration to be favored (not for convenience but to optimize the delivery of the active pharmaceutical ingredients (here, the antibody moiety and/or the cytotoxin)). Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation is also contemplated and can be used to deliver the present conjugates. Thus, the invention provides compositions for parenteral administration that include protein conjugates dissolved or suspended in an acceptable carrier, preferably an aqueous carrier, such as water, buffered water, saline, buffered saline (e.g., PBS), and the like. One or more of the excipients included may help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The pharmaceutical compositions may be sterile; they may be sterilized by conventional sterilization techniques or may be sterile filtered. Aqueous solutions may be packaged for use as is, or lyophilized, the lyophilized preparation, which is encompassed by the invention, being combined with a sterile aqueous carrier prior to administration. The pH of the pharmaceutical compositions typically will be between 3 and 11 (e.g., between about 5 and 9) or between 6 and 8 (e.g., between about 7 and 8). In some embodiments, the pH of the pharmaceutical compositions is between about 7.0 and 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Methods of Treatment:

The pharmaceutical compositions described above can be formulated to include a therapeutically effective amount of a protein conjugate. Therapeutic administration encompasses prophylactic applications. Based on genetic testing and other prognostic methods, a physician in consultation with their patient may choose a prophylactic administration where the patient has a clinically determined predisposition or increased susceptibility (in some cases, a greatly increased susceptibility) to a CNS cancer. The pharmaceutical compositions of the invention can be administered to the subject (e.g., a human patient) in an amount sufficient to delay, reduce, or preferably prevent the onset of clinical disease. In therapeutic applications, compositions are administered to a subject (e.g., a human patient) already suffering from a CNS cancer in an amount sufficient to at least partially improve a sign or symptom or to inhibit the progression of (and preferably arrest) the symptoms of the condition, its complications, and consequences. An amount adequate to accomplish this purpose is defined as a “therapeutically effective amount.” A therapeutically effective amount of a pharmaceutical composition may be an amount that achieves a cure, but that outcome is only one among several that can be achieved. As noted, a therapeutically effect amount includes amounts that provide a treatment in which the onset or progression of the cancer is delayed, hindered, or prevented, or the cancer or a symptom of the cancer is ameliorated. One or more of the symptoms may be less severe. Recovery may be accelerated in an individual who has been treated.

Amounts effective for this use may depend on the severity of the CNS cancer and the weight and general state of the subject, but generally range from about 0.05 μg to about 1000 μg (e.g., 0.5-100 μg) of an equivalent amount of the antibody-polypeptide-cytotoxin conjugate per dose per subject. Suitable regimes for initial administration and booster administrations are typified by an initial administration followed by repeated doses at one or more hourly, daily, weekly, or monthly intervals by a subsequent administration. For example, a subject may receive a protein conjugate in the range of about 0.05 to 1,000 μg equivalent dose as compared to an unconjugated antibody moiety per dose one or more times per week (e.g., 2, 3, 4, 5, 6, or 7 or more times per week). For example, a subject may receive 0.1 to 2,500 μg (e.g., 2,000, 1,500, 1,000, 500, 100, 10, 1, 0.5, or 0.1 μg) dose per week. A subject may also receive a conjugate of the invention in the range of 0.1 to 3,000 μg per dose once every two or three weeks. A subject may also receive 2 mg/kg every week (with the weight calculated based on the weight of the conjugate or the antibody moiety).

The total effective amount of an antibody-polypeptide-cytotoxin conjugate in the pharmaceutical compositions of the invention can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are contemplated.

The therapeutically effective amount of one or more agents present within the compositions of the invention and used in the methods of this invention applied to mammals (e.g., humans) can be determined by one of ordinary skill in the art with consideration of individual differences in age, weight, and other general conditions (as mentioned above). Because the antibody-polypeptide-cytotoxin conjugates of the invention exhibit an enhanced ability to cross the BBB, the dosage of the antibody moiety can be lower than an effective dose of the antibody moiety when unconjugated. For example, the dosage of the antibody moiety can be less than or about 90%, 75%, 50%, 40%, 30%, 20%, 15%, 12%, 10%, 8%, 7%, 6%, 5%, 4%, 3%, 2%, 1%, 0.5%, or 0.1% of the dose of required for a therapeutic effect with the same or a comparable unconjugated agent.

Therapeutically effective amounts can also be determined empirically by those of skill in the art. For example, either single or multiple administrations of the pharmaceutical compositions of the invention can be carried out with dosage levels and the timing or pattern of administration being selected by the treating physician. The dose and administration schedule can be determined and adjusted based on the severity of the disease or condition in the subject, which may be monitored throughout the course of treatment according to the methods commonly practiced by clinicians or other skilled healthcare professionals.

Kits:

The kits of the invention can include any combination of the compositions described above and suitable instructions (whether written and/or provided as audio-, visual-, or audiovisual material). In one embodiment, the kit includes a pharmaceutical composition or a precursor thereto that is packaged together with instructions for use and, optionally, any device useful in manipulating the compositions in preparation for administration and/or in administering the compositions. For example, the kits of the invention can include one or more of: diluents, gloves, vials or other containers, pipettes, needles, syringes, tubing, stands, spatulas, sterile cloths or drapes, positive and/or negative controls, and the like. In another embodiment, the kits of the invention include compositions and reagents useful in making a protein conjugate. For example, the kits can include one or more of: an antibody moiety, a linker, a linker-bound antibody moiety, a polypeptide, a chemical entity reactive with the linker, and/or a modified polypeptide. For example, in one embodiment, a kit can include an antibody moiety, a linker, a chemical entity reactive with the linker, and a polypeptide. In other embodiment, the kit can include a linker-bound antibody moiety and a modified polypeptide. As with the kits useful in administering the compositions, kits useful in making the compositions can include any one or more of: diluents, gloves, vials or other containers, pipettes, needles, syringes, tubing, stands, spatulas, sterile cloths or drapes, positive and/or negative controls, and the like. Any reagents useful in binding a linker to an antibody moiety or polypeptide, modifying a polypeptide, conjugating a linker-bound antibody moiety to a modified polypeptide (or vice versa; conjugating an antibody moiety to a linker-bound polypeptide), or purifying and testing a protein conjugate can also be included. As noted, the linker-bound antibody moieties and modified polypeptides are featured compositions of the invention.

### EXAMPLES

### Example 1

### Synthesis of the Conjugate anti-HER2 mAb-[MFCO-An2-(SuDoce)₂]ₙ

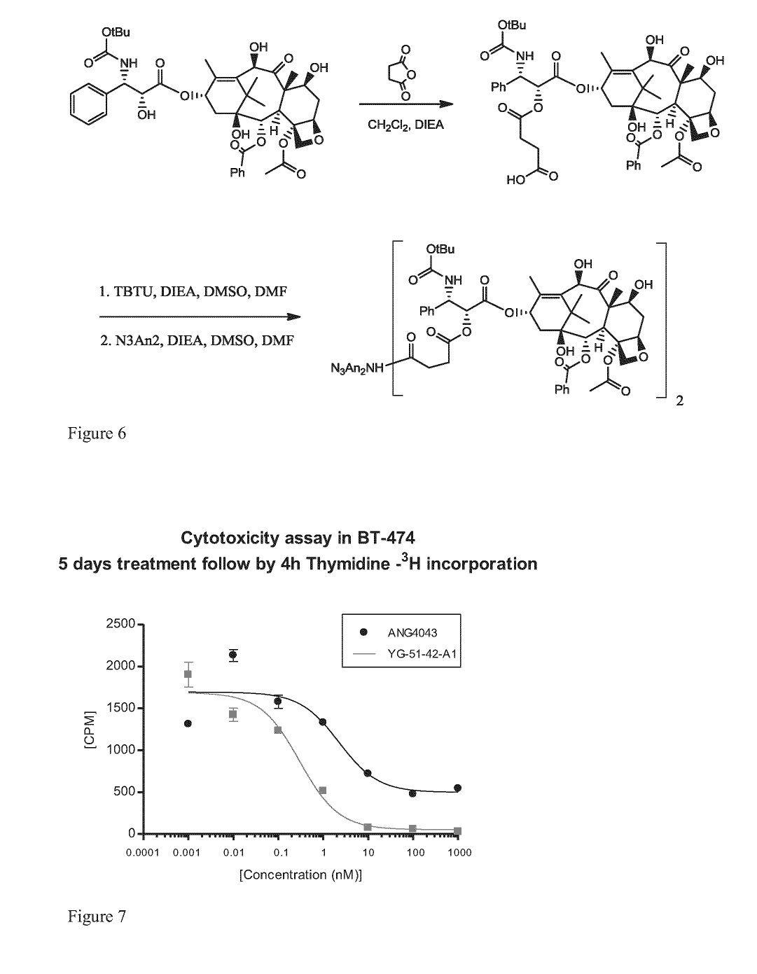

A scheme for conjugating a polypeptide of the invention to a cytotoxic agent is shown in FIG. 6. The scheme exemplifies the conjugation between Angiopep2 (An2) and docetaxel (Doce) to generate N₃An2-(SuDoce)₂. To generate the intermediate DoceSuOH, DIEA (0.21 ml, 1.2 mmol) was added dropwise to a suspension of docetaxel (0.81 g, 1.0 mmol) and succinic anhydride (105 mg, 1.05 mmol) in DCM (7 ml) under stirring. The mixture was stirred at room temperature and monitored by UPLC. After 2 hours, the reaction was complete. The solvent was removed, and the resulting residue was dissolved in DMF (2 ml). The solution was diluted with 30% MeCN in water with 0.05% formic acid (6 ml) and directly loaded onto a phenyl 42 ml column for purification. DoceSuOH was obtained as a white powder (0.68 g, 75%) after lyophilization, UPLC purity >95%. To generate N₃An2-(SuDoce)₂, DIEA (0.012 ml, 0.07 mmol) was added dropwise to a solution of DoceSuOH (31 mg, 0.034 mmol) and HATU (14 mg, 0.037 mmol) in DMF (0.8 ml) at 5° C. under stirring. The mixture was stirred at 5° C. to room temperature for 30 minutes, then a solution of AzidoAngpep-2 (53 mg, 0.017 mmol) in DMSO (0.2 ml) and DMF (0.5 ml) was added. The mixture was stirred at room temperature for 30 minutes. HPLC showed the reaction was complete. The reaction mixture was purified using preparative HPLC (30% to 60% MeCN in H₂O and 0.05% FA) to give N₃An2-(SuDoce)₂ (32 mg, 45%) as white powder after lyophilization, UPLC purity >95%.

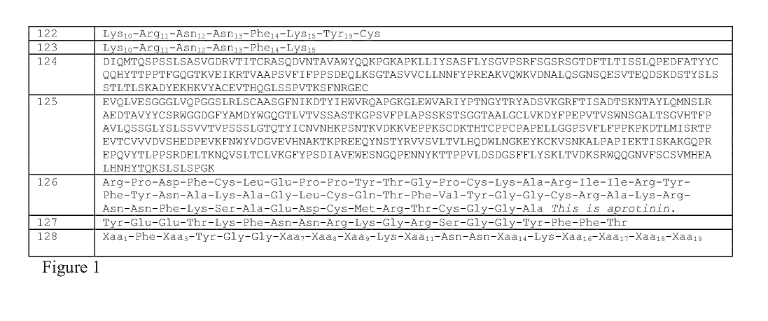

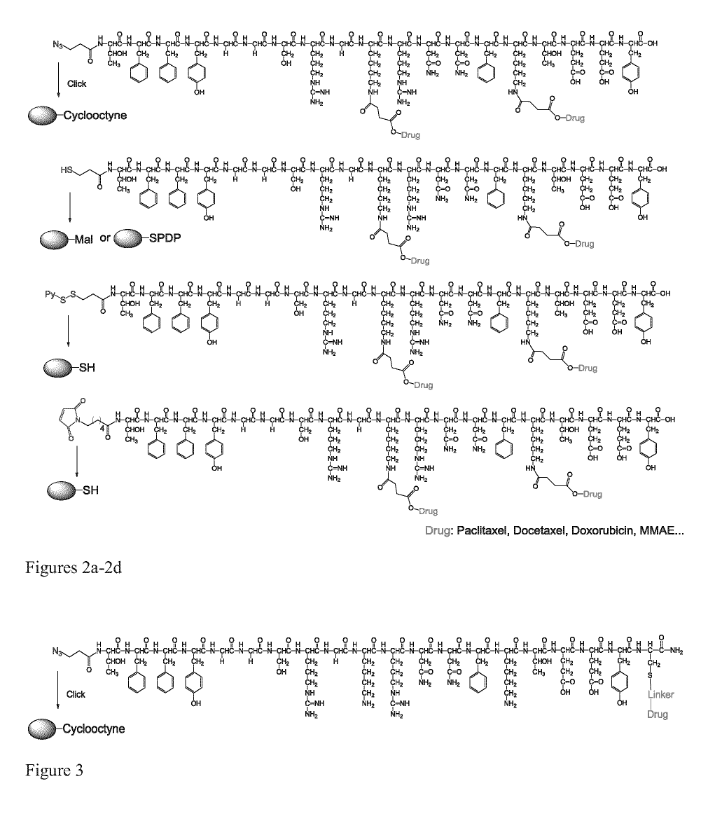

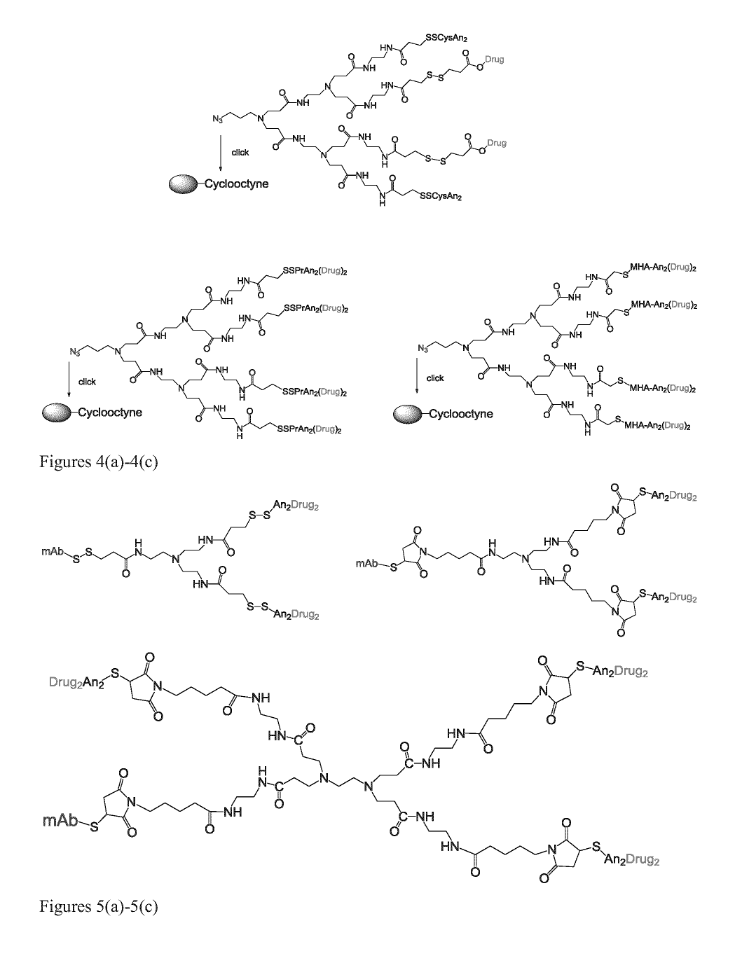

We then conjugated N₃An2-(SuDoce)₂ to an anti-HER2 monoclonal antibody as illustrated in FIGS. 3, 4, and 5. To generate the antibody-linker portion of the conjugate (anti-HER2 mAb-MFCOₙ), MFCO (2.0 mg, 6.9 μmol) was dissolved in DMSO (1 ml), and 0.07 ml (0.48 μmol, 8 eq) of the solution was transferred to a PBS buffer solution of trastuzumab (5 mg/ml, 1.8 ml). The pH of the solution was adjusted to 8.0 using bibasic phosphate solution. The mixture was shaken and allowed to react at room temperature for 3 hours. The modified antibody moiety was purified from excess small molecules using a salt exchange column eluting with 20 mM phosphate buffer pH 7.0. Fractions containing protein were collected, and the buffer was changed to citrate/phosphate buffer pH 5.0 (25 mM, 50 mM respectively) using amicon centrifugal filter (MWCO 10,000). A final solution of 3 ml was obtained, and Bradford assay gave a concentration of 2.7 mg/ml, a 90% yield. The antibody-linker, which we may refer to as activated trastuzumab was then conjugated to the polypeptide-cytotoxin portion of the conjugate to generate an anti-HER2 mAb-[MFCO-An2-(SuDoce)₂]ₙ construct. More specifically, the activated trastuzumab (8.1 mg, 3 ml, 0.046 μmol) was diluted to 8 ml with acetate buffer (pH 5) and tween 80 (0.008 ml) was added and vertexed to make a homogenous solution. A solution of N₃An2(DoceSu)₂ (2.2 mg, 8 eq) in DMSO (0.3 ml) was added at room temperature. The mixture was shaken and stored at room temperature for 2 days. The excess of small molecules was removed using an amicon centrifugal filter (MWCO 10,000) and citrate/phosphate buffer pH 5.0 (25 mM, 50 mM respectively) 4 times. A final solution of 3 ml was obtained, concentration 2.8 mg/ml, quantitative Maldi-tof analysis showed a mass of 162000, which indicated around 3 molecules of N₃An2-(SuDoce)₂ was conjugated to the anti-HER2 mAb.

### Example 2

### Cytotoxicity Assay

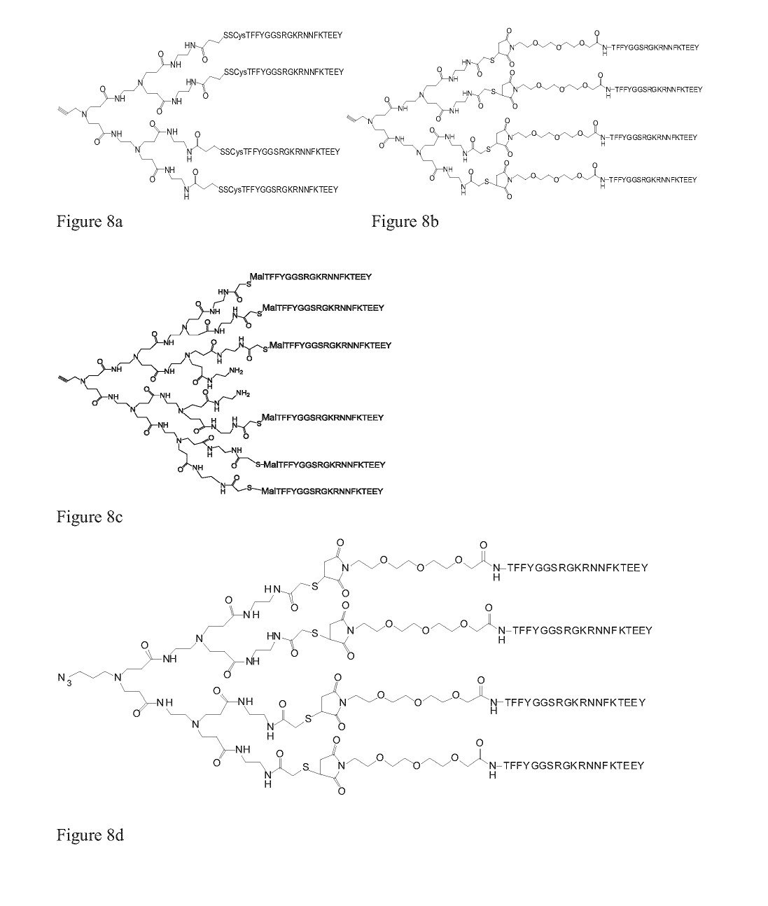

Cell proliferation using thymidine incorporation assay. BT-474 tumor cells were cultured in white 96-well plates (Perkin Elmer, USA) at a density of 7500 cells per well. First, cells were synchronized 24 h in serum deprived medium. After incubation of cells with increasing concentrations of anti-HER2-Angiopep-2 conjugate (ANG4043) or anti-HER2-Angiopep-2-(Docetaxel)2 for 5 days, the complete medium was aspirated and cells were pulse labeled for 4 h at 37° C./5% CO2 with complete medium containing 2.5 μCi/mL [methyl-3H]-thymidine (Perkin Elmer, USA). Cells were washed, fixed and dried before addition of the scintillation liquid Microscint 0 from Perkin Elmer. After 24 h, plates were read using a plate reader TopCount (Perkin Elmer, USA) for determination of tritium uptake. Incorporated [3H]-thymidine was plotted for each drug concentrations. The results are illustrated in FIG. 7.

What is claimed is:

1. A protein conjugate comprising an antibody moiety, a polypeptide, and a cytotoxic agent, wherein the polypeptide comprises the amino acid sequence Lys-Arg-Asn-Asn-Phe-Lys (SEQ ID NO:123) or a biologically active analog thereof.

2. The conjugate of claim 1, further comprising a linker between the antibody moiety and the polypeptide and/or between the polypeptide and the cytotoxic agent.

3. The conjugate of claim 2, wherein the linker is a homofunctional linker or a heterofunctional linker.

4. The protein conjugate of claim 3, wherein the homofunctional linker is a homobifunctional, homotrifunctional, or homotetrafunctional linker comprising two, three, or four reactive groups, respectively, that react with a primary amine, a thiol group, a hydroxyl group, or a carbohydrate, and the heterofunctional linker is a heterobifunctional, heterotrifunctional, or heterotetrafunctional linker comprising at least one reactive group that reacts with a primary amine, a thiol group, a hydroxyl group, or a carbohydrate.

5. (canceled)

6. The protein conjugate of claim 2, wherein the linker is a monofluoro cyclooctyne (MFCO), bicyclo[6.1.0]nonyne (BCN), dibenzocyclooctyne (DBCO), N-succinimidyl-S-acetylthioacetate (SATA), N-succinimidyl-S-acetylthiopropionate (SATP), or N-Hydroxy-succinimide (NETS).

7. The conjugate of claim 1, wherein the polypeptide comprises the amino acid sequence Thr₁-Phe₂-Phe₃-Tyr₄-Gly₅-Gly₆-Cys₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉ (SEQ ID NO:67) or an analog thereof or Thr₁-Phe₂-Phe₃-Tyr₄-Gly₅-Gly₆-Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉ (SEQ ID NO:97) or an analog thereof.

8-9. (canceled)

10. The conjugate of claim 1, wherein the analog comprises the sequence Phe₃-Tyr₄-Gly₅-Gly₆-Cys₇/Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:118); Gly₅-Gly₆-Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:119); Ser₇-Arg₈-Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:120); Gly₉-Lys₁₀-Arg₁₁-Asn₁₂-Asn₁₃-Phe₁₄-Lys₁₅-Thr₁₆-Glu₁₇-Glu₁₈-Tyr₁₉-Cys (SEQ ID NO:121); or Lys₁₀-Arg₁₁-Asn₁₂-Asn₁₃-Phe₁₄-Lys₁₅-Tyr₁₉-Cys (SEQ ID NO:122).

11. The conjugate of claim 1, wherein the polypeptide comprises at least one amino acid residue in the D-form.

12. The conjugate of claim 1, wherein the conjugate comprises 1-10 polypeptides and one antibody moiety.

13. (canceled)

14. The conjugate of claim 1, wherein the polypeptide and the cytotoxic agent are present in a ratio of 1:1 to 1:3 (polypeptide:cytotoxic agent).

15. The conjugate of claim 1, wherein each polypeptide is linked, via at least one linker, to an antibody moiety.

16. The conjugate of claim 1, wherein the antibody moiety is a tetrameric antibody or a biologically active variant thereof.

17. The conjugate of claim 1, wherein the antibody moiety is a single chain antibody (scFv), Fab fragment, or F(ab′)2 fragment.

18. The conjugate of claim 1, wherein the antibody moiety comprises a human, chimeric or humanized antibody or a biologically active variant thereof.

19-28. (canceled)

29. The conjugate of claim 1, wherein the antibody moiety is an anti-cancer agent or an anti-inflammatory agent.

30. The conjugate of claim 1, wherein the cytotoxic agent is a taxane, an alkaloid, an anthracycline, an auristatin, an antifolate, a calicheamicin, a duocarmycin, a mitomycin, a pyrimidine analog, or a derivative of mytansine.

31. (canceled)

32. The conjugate of claim 30, wherein the alkaloid is a vinca alkaloid; the anthracycline is doxorubicin; the auristatin is monomethyl auristatin E (MMAE); the antifolate is methotrexate or aminopterin; the calicheamicin is calicheamicin γ 1; the duocarmycin is adozelesin, bizelesin, or carzelesin; the mitomycin is mitomycin C; the pyrimidine analog is fluorouracil; and the derivative of mytansine is a mytansinoid.

33. (canceled)

34. The conjugate of claim 1, wherein the antibody moiety, the polypeptide, and the cytotoxic agent are linked in a linear conjugate.

35. The conjugate of claim 1, wherein the antibody moiety, the polypeptide, and the cytotoxic agent are linked in a dendrimeric conjugate.

36. A pharmaceutical composition comprising the conjugate of claim 1 and a pharmaceutically acceptable carrier.

37. (canceled)

38. A method of treating a patient who is suffering from cancer, the method comprising:
identifying a patient in need of treatment; and
administering to the patient a therapeutically effective amount of the pharmaceutical composition of claim 36.

39-45. (canceled)
